(19) United States
(12) Patent Application Publication
(10) Pub. No.: US 2023/0332108 A1
(43) Pub. Date: Oct. 19, 2023

(54) ENDOTHELIAL CELLS AND METHODS OF MAKING AND USING

(71) Applicant: University Health Network, Toronto (CA)

(72) Inventors: Gordon Keller, Toronto (CA); Blair Kenneth Gage, Toronto (CA)

(21) Appl. No.: 18/181,889

(22) Filed: Mar. 10, 2023

Related U.S. Application Data
(60) Provisional application No. 63/318,556, filed on Mar. 10, 2022.

(51) Int. Cl. A61P 1/16; C12N 5/071

(52) U.S. Cl. CPC C12N 5/069; A61P 1/16; C12N 2501/155; C12N 2501/16; C12N 2506/45

(57) ABSTRACT

This disclosure describes vascular endothelial cells (VECs) and liver sinusoidal endothelial cells (LSECs), and methods and compositions for producing such cells.

### TECHNICAL FIELD

This disclosure generally relates to endothelial cells and methods of making and using such cells.

### CROSS REFERENCE TO RELATED APPLICATIONS

This application claims the benefit of priority under 35 U.S.C. § 119(e) to U.S. Application No. 63/318,556 filed on Mar. 10, 2022.

### BACKGROUND

The endothelium of the liver is heterogenous and carries out many functions essential for organ homeostasis. The liver sinusoidal endothelial cells (LSECs) that line the sinusoids of the organ represent a critical component of this endothelium, as they perform many organ-specific functions including passive blood component transport through fenestrations and active scavenging of circulating biomolecules and pathogens. LSECs also secrete several functional proteins including a number of ‘angiocrines’ that regulate hepatic regeneration as well as coagulation factor VIII (FVIII) that is critical for proper hemostasis.

Given this diverse range of activities, LSECs are directly or indirectly involved in many liver diseases. Disruption of FVIII production or activity results in the bleeding disorder Hemophilia A, whereas alterations in other LSEC functions are observed in metabolic diseases such as NAFLD and NASH. LSEC functions also are acutely altered during drug-induced liver injury and in conditions of chronic liver damage such as cirrhosis and liver cancer. Given their central role in normal liver physiology and disease processes, LSECs represent an important target population for developing new therapeutic interventions to treat some of the most devastating forms of these diseases.

### SUMMARY

As access to human LSECs is limited, recent efforts have focused on generating these cells from mouse and human pluripotent stem cells (hPSCs). It previously was shown that hPSC-derived angioblasts that displayed a venous phenotype could generate LSECs more rapidly than arterial-like cells following transplantation into recipient NSG mice. These observations were consistent with lineage tracing studies in mice and zebrafish, showing that LSECs derive from the developing venous vasculature. Although these studies identified the vascular subtype from which LSECs originate, the early developmental steps leading to the generation of this lineage (e.g., mesoderm induction) are not well understood.

The importance of mesoderm induction to subsequent lineage commitment is highlighted by the findings from lineage tracing in mice, which revealed that cell fate decisions are made during the time of gastrulation. For example, in the heart, ventricular and atrial cardiomyocytes are generated from different subpopulations of Mesp1-expressing mesoderm that are specified at different times. Similarly, different human heart and blood cell lineages are specified at the mesoderm stage during hPSC differentiation.

This disclosure describes a protocol for the generation of human pluripotent stem cell (hPSC)-derived venous endothelial cells (VECs) that display robust potential to engraft the liver of either normal or Hemophilia ANSG recipient mice, where the cells then differentiated into functional liver sinusoidal endothelial cells (LSECs). The experimental design uses a novel competitive repopulation assay to show that the efficiency of LSEC engraftment is directly related to the subtype of mesoderm induced in the cultures. The work described herein demonstrates that mesoderm characterized by the co-expression of KDR and CD235a/b (i.e., having a phenotype of KDR+ CD235a/b+) induced through optimal levels of BMP and Activin signaling generates a VEC population that displays up to 50-fold greater engraftment potential than venous angioblasts derived from expression of KDR and the lack of expression of CD235a/b (i.e., having a phenotype of KDR+ CD235a/b-) mesoderm generated with the previously described protocol. With these engraftment efficiencies, nearly all (>80%) of the murine sinusoidal endothelium can be replaced with human LSECs. In addition to producing a VEC population with dramatically higher LSEC potential than previous protocols, the protocols described in this disclosure are approximately 10-fold more efficient, yielding about 1 VEC per input hPSC.

The levels of human vascular engraftment achieved with the approach described herein are the highest reported to date. In addition, the levels are therapeutically relevant as it is demonstrated herein that the LSECs produced after engraftment were able to produce sufficient levels of FVIII to reverse the bleeding phenotype in Hemophilia A recipients. With these efficiencies, the methods and compositions described herein provide commercial applications for the development of vascular cell therapies for Hemophilia A and other liver diseases, as well as for ischemic diseases that affect the heart and skeletal muscle.

In one aspect, methods of making mesodermal cell-derived venous endothelial cells (VECs) are provided. Such methods typically include providing human pluripotent stem cells (hPSCs) having a pluripotent phenotype indicative of human induced pluripotent stem cells (iPSCs) or human embryonic stem cells (hESCs); contacting the hPSCs with modulators of BMP and Activin signaling under appropriate conditions to produce mesodermal cells having a phenotype of KDR+ and CD235a/b+; and inducing the mesoderm cells having the phenotype of KDR+ and CD235a/b+ to differentiate into mesodermal cell-derived VECs.

In some embodiments, the contacting step is performed in embryoid bodies (EBs). In some embodiments, the contacting step is performed in a monolayer. In some embodiments, the inducing step is performed in embryoid bodies (EBs). In some embodiments, the inducing step is performed in monolayer adherent form.

Representative modulators of BMP signaling include, without limitation, BMP4, BMP2, or small molecule BMP signaling agonists (e.g., ventromorphins [PMID 28787124], ID1 or ID2 [PMID 23527084], chromenone 1 [PMID 35108017], SB 4 [CAS number 100874-08-6] or similar). Representative modulators of Activin signaling include, without limitation, Activin A, NODAL, or small molecule TGFbeta signaling agonists.

In some embodiments, the mesoderm cells also have a phenotype of PDGFRa+, CD56+ and APLNR+. In some embodiments, the mesodermal cell-derived VECs have a phenotype of CD34+, CD31+, CD73+, and CD184-. In some embodiments, the mesodermal cell-derived VECs also have a phenotype of NRSF2+, NRP2+, NT5E+ and EPHB4+.

In some embodiments, the inducing takes place in the presence of VEGF-A, VEGF-B, VEGF-C, VEGF-D or PIGF, or small molecule VEGFA signaling agonists. In some embodiments, the inducing takes place in the presence of bFGF/FGF2 or small molecule FGF signaling agonists.

In some embodiments, VECs engraft in vivo and mature to liver sinusoidal endothelial cells (LSECs). In some embodiments, the VECs differentiate into functional liver sinusoidal endothelial cell-like cells (LSEC-LCs) having a phenotype of CD31+, CD32+ and LYVE1+. In some embodiments, the VEC-derived-LSECs further have a phenotype of CD32B+, STAB2+ and FVIII+.

In some embodiments, the LSECs express FCGR2B, LYVE1, STAB2, F8, CD14, MRC1, CD36 and RAMP3 at amounts that are greater than VEC-derived non-LSEC endothelium with a phenotype of CD31+ CD32- LYVE1-. In some embodiments, the LSECs express PECAM1, VWF and CALCRL at amounts that are less than VEC-derived non-LSEC endothelium with a phenotype of CD31+, CD32- LYVE1-. In some embodiments, the mesoderm cells generate a population of VECs that exhibits at least a 20-fold greater (e.g., 30-fold, 40-fold, 50-fold greater) engraftment potential than venous angioblast cells derived from mesoderm cells having a phenotype of KDR+ and CD235a/b-.

In another aspect, methods of treating a subject suffering from a liver disease are provided. Such methods typically include administering / delivering the cells of any one of the preceding claims to the subject, wherein the cells efficiently engraft in the liver, thereby treating the subject suffering from the liver disease.

In some embodiments, the liver disease is Hemophilia A or other monogenic endothelial disease. In some embodiments, the liver disease is acute drug liver injury (e.g., Sinusoidal Obstruction Syndrome (human monocrotaline toxicity), or Acetaminophen overdose), chronic liver injury (e.g., NASH, NAFLD, Cirrhosis, chronic drug injury), liver cancer (e.g., primary, hepatocellular carcinoma; or secondary (e.g., colon, breast, pancreatic) liver metastatic cancer).

In some embodiments, the engrafted cells differentiate into LSECs. In some embodiments, the LSECs express therapeutic amounts of FVIII (e.g., sufficient levels of FVIII to reverse the severe bleeding phenotype in a subject suffering from Hemophilia A).

In some embodiments, the administering / delivering step is repeated more than once. In some embodiments, the administering / delivering step comprises intravenous delivery (e.g., via infusion via the hepatic portal vein or systemically).

In some embodiments, the hPSCs, mesoderm cells, VECs or LSECs are rendered hypoimmunogenic through genetic addition or removal of immunoregulatory antigens, resulting in cell populations that are not readily rejected by the recipient despite incomplete immunological matching. In some embodiments, administering / delivering functional LSEC progenitors slows or reverses disease progression.

In some embodiments, the hPSCs are genetically modified such that the VECs have designer or specialized new functions (e.g., where novel exogenous therapeutic proteins are expressed to provide novel therapeutic applications). The list of such exogenous proteins is virtually endless.

Unless otherwise defined, all technical and scientific terms used herein have the same meaning as commonly understood by one of ordinary skill in the art to which the methods and compositions of matter belong. Although methods and materials similar or equivalent to those described herein can be used in the practice or testing of the methods and compositions of matter, suitable methods and materials are described below. In addition, the materials, methods, and examples are illustrative only and not intended to be limiting. All publications, patent applications, patents, and other references mentioned herein are incorporated by reference in their entirety.

### DESCRIPTION OF DRAWINGS

FIGS. 1A-1M show experimental data demonstrating the optimization of mesoderm induction and endothelial specification.

FIG. 1A is a schematic representation of venous endothelial cell differentiation protocol; day 1-4 as suspension embryoid bodies and day 4-8 as monolayer culture.

FIG. 1B shows representative flow cytometric analysis of day 4 mesoderm populations (KDR⁺ CD235a/b^{+/-}) and early endothelial lineage cells (KDR⁺ CD34⁺). Concentrations (ng/ml) of BMP4 (“B”) and Activin A (“A”) used for mesoderm specification are indicated.

FIG. 1C shows cell numbers from induction with different concentrations (ng/ml) of BMP4 and Activin A (ACTA) at day 4, normalized to the number of generated from induction with 3B0A, n=3. See also FIG. 9.

FIGS. 1D-1F are graphs showing day 4 flow cytometric analyses of indicated markers in mesoderm populations induced with different concentrations of BMP4 and ACTA, n=5-6.

FIGS. 1G-1I show data from RT-qPCR analyses of expression in indicated genes in day 4 bulk mesoderm populations, n=3.

FIGS. 1J-1K show representative flow cytometric analysis of endothelial (CD34⁺ CD31⁺), venous EC-like (CD34⁺ CD31⁺ CD73⁺ CD184⁻) or arterial EC-like (CD34⁺ CD31⁺ CD73⁺ CD184⁺) markers on monolayer populations generated by 4 days of adherent culture from mesoderm induced as indicated. EC conditions: venous (10 ng/ml VEGF-A, 30 ng/ml bFGF), arterial (100 ng/ml VEGF-A, 30 ng/ml bFGF).

FIG. 1L shows flow cytometric based quantification of the frequency of endothelial cells (CD34⁺ CD31⁺) in day 8 monolayer populations generated under venous EC conditions from mesoderm generated with the indicated amounts BMP4 and ACTA. ‘Artery’ indicates 12B4A mesoderm in arterial EC conditions, n=3.

FIG. 1M shows the total day 8 venous EC yield from K⁺2⁺ mesoderm generated under the indicated conditions, numbers are normalized the number generated from mesoderm induced with 3B0A. Total yield is calculated as day 8 venous-like EC CD34⁺ CD31⁺ CD73⁺ CD184⁻frequency × day 8 total cells × day 4 KDR⁺ CD235a/b⁺ frequency × day 4 total cells and normalized to 3B0A condition, n=3. * indicates p<0.05 comparing 12B4A to 12B0A, 12B1A, 12B2A, 12B8A, 12B16A, 3B4A, 6B4A, and 24B4A by ANOVA with Bonferroni post-hoc testing. Heat map data are represented as mean values with individual points and mean ± SEM depicted in FIG. 9. Black/white bold outline surrounding 6B4A, 6B8A, 12B4A, 12B8A in ‘C-I, L-M’ indicates stable optimal conditions that give rise to KDR⁺ CD235a/b⁺ mesoderm that efficiently generates venous endothelium. For RT-qPCR analysis, expression values are normalized to levels of the housekeeping gene TBP.

FIGS. 2A-2I shows the characterization of 12B4A-induced mesoderm.

FIG. 2A shows representative flow cytometric analysis of KDR and CD235a/b expression in a day 4 12B4A-induced mesoderm population.

FIG. 2B shows representative flow cytometric analysis of CD34 and CD31 expression on VEC monolayer populations before and after CD34 MACS enrichment and of CD184 and CD73 expression on the CD34⁺enriched cells.

FIG. 2C shows cell yield at indicated stages of lineage development (CD34⁺: MACS enriched). Mean values ± SEM normalized to hESC input (1) are shown. ANOVA; n=5-20; *p<0.05, ***p<0.001 between protocols at indicated protocol stage.

FIG. 2D shows flow cytometric analyses of indicated surface markers on day 4 12B4A-induced mesoderm, n=11.

FIG. 2E shows flow cytometric analyses of CD34 on day 8 populations generated from the indicated 12B4A day 4 mesoderm subsets. ‘Pre’: prior to MACS isolation, ‘34⁺’: CD34⁺ MACS-enriched fraction, n=4-6.

FIG. 2F shows flow cytometric analyses of the expression of venous-like (CD34⁺ CD31⁺ CD73⁺ CD184⁻) or arterial-like (CD34⁺ CD31⁺ CD73⁺ CD184⁺) endothelial markers on day 8 CD34⁺ MACS enriched populations generated from indicated day 4 mesoderm subsets, n=4-5.

FIG. 2G shows RT-qPCR analyses of indicated genes in unsorted (UNS) or FACS purified day 4 mesoderm populations induced with either 10BOA3C or 12B4A. Mean TBP normalized expression levels are depicted as increasing colours from blue (low) to red (high) scaled for each gene. ANOVA; n=3-8, *p<0.05 comparing 10BOA3CUNS with indicated sample. See also FIG. 10.

FIG. 2H shows RT-qPCR quantification of expression of indicated genes in populations generated from the 12B4A induced day 4 mesoderm unsorted (white) or K⁺2⁻ (red) and K⁺2⁺ (magenta) subsets on different days of culture. ‘34⁺’ day 8 cells are CD34⁺ MACS enriched, n=4.

FIG. 21 shows RT-qPCR quantification of venous endothelial marker gene expression in day 8 VECs generated from the indicated day 4 mesoderm subtypes in the presence of 10 ng/ml VEGF-A, 30 ng/ml bFGF. Arterial cells were generated from unsorted 12B4A induced mesoderm in the presence of 100 ng/ml VEGF-A, 30 ng/ml bFGF. ANOVA; n=4-7, *p<0.05, ***p<0.001 as indicated. All data are represented as mean ± SEM. For RT-qPCR analysis, expression values are normalized to levels of the housekeeping gene TBP.

FIGS. 3A-3I shows LSEC engraftment in neonatal and adult NSG recipients.

FIG. 3A is a schematic representation of VEC differentiation and transplantation in neonatal or adult NSG mice.

FIGS. 3B-3C shows individual and cumulative engraftment (GFP or RFP⁺% of NPC) of VEC, in neonate (Neo) and adult recipients at the indicated times. VECs were generated from day 4 12B4A induced unsorted ‘UNS’ or K⁺2⁺ mesoderm, n=2-13. Black line at 1% engraftment denotes graft size that shows detectable FVIII antigen. See also FIG. 11.

FIG. 3D shows representative flow cytometric analysis of a liver NPC population from a neonate and adult recipient.

FIG. 3E shows the quantification of human endothelial (GFP⁺ CD31⁺) and LSEC (GFP⁺ CD31 ⁺ CD32⁺ LYVE1⁺) engraftment of K⁺2⁺ derived VECs in neonatal and adult recipients between 46 and 123 days post-transplant (neonatal Tx: n=13, adult Tx: n=8).

FIG. 3F shows RT-qPCR analysis of LSEC markers in indicated FACS isolated populations from adult recipients transplanted with K⁺2⁺ derived VECs (TBP normalized mean ± SEM, ANOVA; n=6-7, *p<0.05, **p<0.01, ***p<0.001, vs GFP⁺ CD31⁺ cells).

FIG. 3G is histological analysis showing the presence of CD31⁺, LYVE1⁺, CD32B⁺, STAB2⁺, and Ku80⁺ cells in the livers of adult recipients engrafted with K⁺2⁺ derived VECs. “*” indicates location of enlarged inset panels; scale bar represents 60 µm.

FIG. 3H is immunofluorescent analysis of the liver of an adult recipient of K⁺2⁺ derived VECs. Transplanted cells are shown as GFP (green), FVIII expressing cells as red and all human cells (Ku80) as white. DAPI counterstain (blue) marks all nuclei in the section. “*” indicates location of enlarged single channel montage panel; scale bar represents 50 µm. Central vein (CV) and portal vein (PV) are indicated when present in images.

FIG. 3I shows human specific FVIII antigen levels (IU/ml, 1 IU/ml =100% normal human plasma levels) in plasma of mice transplanted with K⁺2⁺ derived VECs (n=3-10, mean ± SEM, ANOVA, ***p<0.001, as indicated).

FIGS. 4A-4I shows competitive transplantation of liver vasculature progenitors. All data are presented as mean ± SEM.

FIG. 4A is a schematic representation of the competitive liver vascular transplantation strategy using CD34⁺ cells from congenic eGFP and tdRFP variants of the HES2 hESC line. Mixtures of RFP⁺ and GFP⁺ VECs are transplanted into adult recipients, the ratio of RFP⁺ and GFP⁺ human cells and LSECS is analyzed 40+ days following transplantation.

FIG. 4B shows flow cytometric analyses of the NPC fraction of the livers of mice transplanted with the indicated ratios of RFP⁺ and GFP⁺ VECs generated from 12B4A-induced mesoderm. Upper row indicates ratio in pre-transplant population and lower row shows ratio in the liver graft (n=3-4 per mix, day 42-43 pTx). See also FIG. 14.

FIG. 4C shows quantification of the RFP⁺ and GFP⁺ component of the graft in the different recipients.

FIG. 4D shows the proportion of the indicated cell types in the grafts generated with the different ratios of GFP⁺ and RFP⁺ VECs.

FIG. 4E shows flow cytometric analyses of the NPC fraction of livers of mice transplanted with the indicated mixtures of day 8 venous angioblasts generated from 10BOA3C-induced mesoderm (Gage et al., 2020), n=5 per mix, day 41-42 pTx.

FIG. 4F shows the quantification of the GFP⁺, RFP⁺, CD31⁺ and indicated LSEC marker positive cells in the NPC fraction of the livers of mice transplanted with the indicated ratios of angioblasts.

FIG. 4G shows linear regression of angioblast derived GFP:RFP graft size from FIGS. 4E and 4F by mixture, n=5.

FIG. 4H shows flow cytometric analyses of the livers of mice engrafted with an equal (50:50) mixture of GFP⁺ day 8 VECs and RFP+ angioblasts. VECs were generated from K⁺2⁺ day 4 12B4A-induced mesoderm and angioblasts from day 4 10B0A3C-induced mesoderm.

FIG. 4I shows the quantification of the GFP⁺, RFP⁺, CD31⁺ and indicated LSEC marker positive cells in the NPC fraction of the livers of mice from two independent experiments (Cohorts) (Cohort 1: n=5, Cohort 2: n=4, day 42-43 pTx).

FIGS. 5A-5I shows competitive transplantation comparing mesoderm and endothelial specification. All data are presented as mean ± SEM.

FIG. 5A is a schematic representation of hybrid differentiation protocols. Arm 1: comparison of mesoderm induction: RFP⁺ 10BOA3C-induced mesoderm vs GFP⁺ 12B4A-induced mesoderm. VECs from both mesoderm populations were generated in monolayer cultures; Arm 2: Comparison of endothelial specification: EB vs monolayer generation of angioblasts and VECs from GFP⁺ and RFP⁺ 12B4A-induced mesoderm. In both experiments, CD34⁺ cells were isolated by MACS sorting and transplanted in 50R:50G ratios.

FIG. 5B shows Arm 1: flow cytometric analysis of KDR and CD235a/b expression on day 4 mesoderm and of CD34, CD31, CD184, and CD73 expression on the day 8 VECs.

FIG. 5C shows flow cytometric analyses of the pre-transplant population (Pre Tx) and of the liver NPC fraction of an engrafted mouse > 40 days post-transplant. Far right graph shows quantification of the per cent of RFP⁺ and GFP⁺ cells in the NPC fraction of livers from recipients in both cohorts (Cohort 1: n=5, Cohort 2: n=5, day 41-44 pTx).

FIG. 5D shows Arm 2: Flow cytometric analysis of the day 4 mesoderm and day 8 angioblasts (upper panel) and VECs (lower panel) of the markers detailed in ‘B’.

FIG. 5E shows flow cytometric analyses of the pretransplant population (Pre Tx) and of the liver NPC fraction of an engrafted mouse > 40 days post-transplant. Far right graph shows quantification of the per cent of RFP⁺ and GFP⁺ cells in the NPC fraction of livers from recipients in both cohorts (Cohort 1: n=5, Cohort 2: n=5, day 41-44 pTx).

FIGS. 5F-5G shows production yield analysis for Arm 1: 10B0A3C Monolayer (FIG. 5F) and Arm 2: 12B4A-EB (FIG. 5G) at indicated stages with mean values above graph, n=3-4.

FIG. 5H show flow cytometric analyses of the pre-transplant population (Pre Tx) and of the liver NPC fraction of an engrafted mouse transplanted (41 days) with an equal mixture of VECs generated from day 4 FACS purified K⁺2⁺ (GFP) and K⁺2⁻(RFP) mesoderm induced with 12B4A. Graph on right shows quantification of the per cent of RFP⁺ and GFP⁺ cells in the NPC fraction of livers from recipients in both cohorts (Cohort 1: n=5, Cohort 2: n=3, day 41 pTx). Ratio of GFP⁺ to RFP⁺ cells is shown below graph.

FIG. 51 shows flow cytometric analyses of the pre-transplant population (Pre Tx) and of the liver NPC fraction of an engrafted mouse transplanted (41 days) with an equal mixture of VECs generated from day 4 FACS purified K⁺2⁺ (GFP) and unfractionated (RFP) mesoderm induced with 12B4A. Graph on right shows quantification of the percent of RFP⁺ and GFP⁺ cells in the NPC fraction of livers from recipients in both cohorts. Ratio of GFP⁺ to RFP⁺ cells is shown below graph (Cohort 1: n=4, Cohort 2: n=3, day 41 pTx).

FIGS. 6A-6H shows the correction of Hemophilia A bleeding by transplantation of venous endothelial cells.

FIG. 6A shows activated partial thromboplastin time (aPTT) in cohort 1 (1.5 × 10⁶ GFP⁺ VECs per Tx) over time compared to NSG-HA mice receiving no cells (No Tx). Mean ± SEM, No Tx n=3, Intraportal Tx (IP) n=2-5, Intrasplenic Tx (IS) n=4. hFVIII activity is relative to recombinant full-length human FVIII (Kovaltry) in IU/ml where 1 IU/ml=100% normal human levels.

FIG. 6B shows blood loss in cohort 1 recipients at 12 weeks post-transplant compared to untransplanted NSG-HA and control C57B16 mice. Blood loss was measured using the tail clip bleeding challenge. Mean ± SEM, ANOVA, n=2-4, **p<0.01, ***p<0.001, as indicated.

FIG. 6C shows aPTT in Cohort 2 (3.0 × 10⁶ RFP⁺ VECs per Tx) over time. Mean ± SEM, IP n=4-5, IS n=6.

FIG. 6D shows blood loss in Cohort 2 recipients at 16 weeks following transplantation compared to untransplanted NSG-HA and wild type NSG mice. Mean ± SEM, ANOVA, n=3-6, **p<0.01, ***p<0.001, as indicated.

FIG. 6E shows human specific circulating FVIII antigen levels (IU/ml, 1 IU/ml =100% normal human plasma levels) in plasma from untransplanted control mice (NSG WT and NSG-HA) and from Cohort 1 (12 weeks pTx) and 2 (16 weeks pTx) recipient mice (mean ± SEM, ANOVA, n=2-9, ^{#}_{′}*p<0.05, ***p<0.001, as indicated).

FIG. 6F shows immunofluorescent analysis of VEC engraftment in the livers of transplanted NSG-HA mice (Cohort 1 at 12-weeks) showing total donor cells in green (GFP), FVIII producing cells in red, human Ku80⁺ cells in white. DAPI counterstain (blue) was used to mark all nuclei in the section.

FIG. 6G shows immunofluorescent analysis of sinusoidal VEC engraftment in the livers of NSG-HA mice (cohort 2 at 16-weeks) showing Ki67⁺ cells in green and human Ku80⁺ cells in red. DAPI counterstain (blue) was used to mark all nuclei in the section.

FIG. 6H shows histological analysis of the livers of Cohort 1 (12 weeks) and 2 (16 weeks) recipients for human endothelial/ LSEC markers including CD31, LYVE1, CD32B, STAB2 and Ku80. “*” indicates location of enlarged inset panels; scale bar represents 50 µm in FIGS. 6F and 6G, and 60 µm in FIG. 6H. Central vein (CV) and portal vein (PV) are indicated when present. See also FIG. 15.

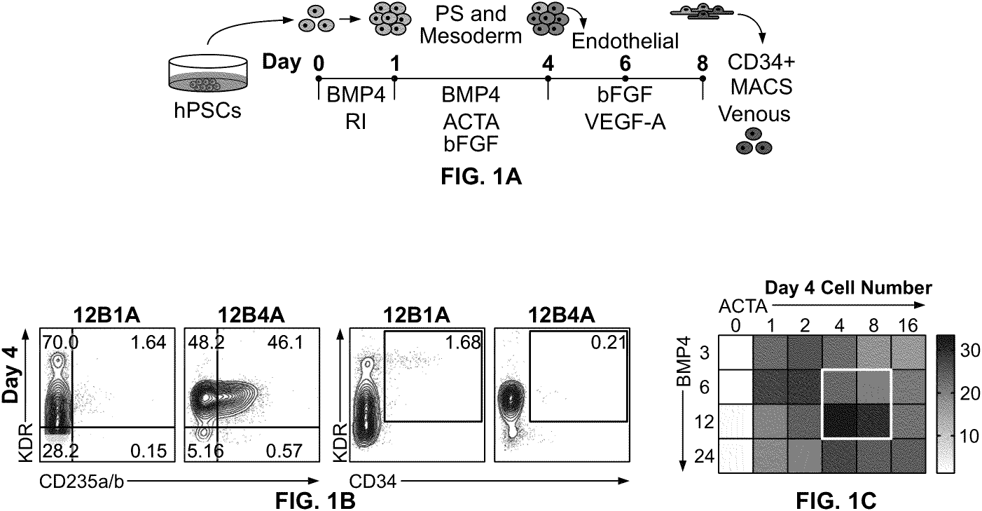

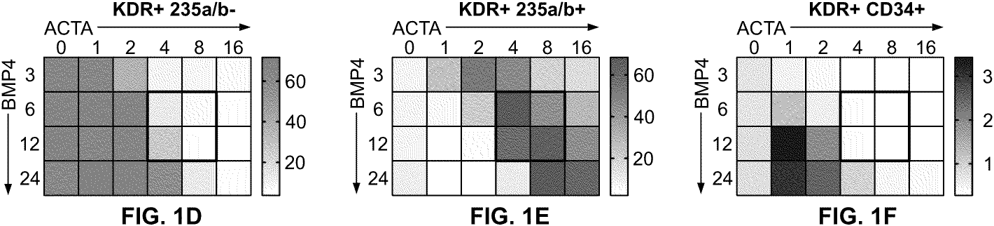

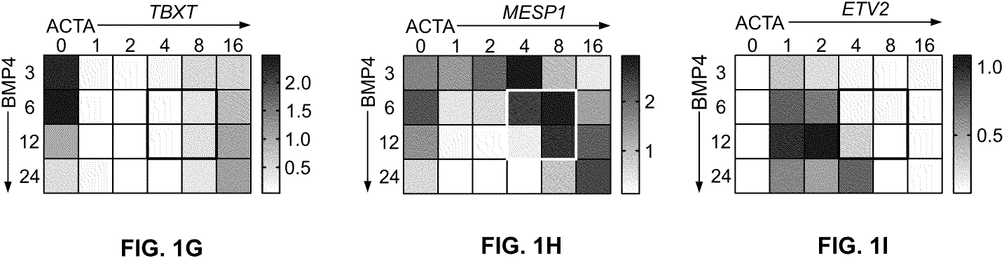

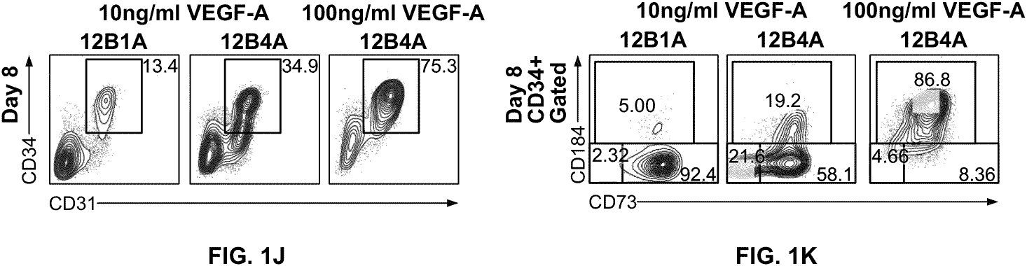

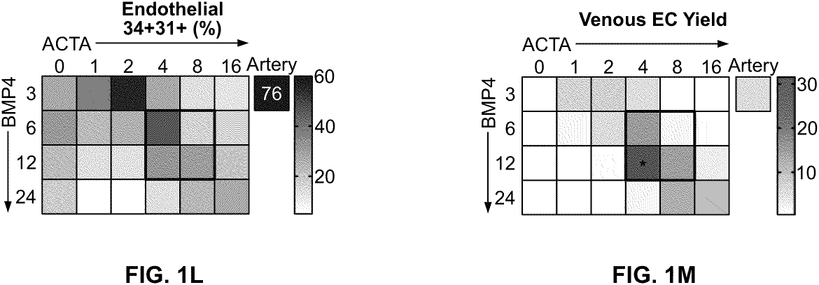

FIGS. 7A-7B shows representative mesoderm BMP4/Activin A grid (related to FIG. 1).

FIG. 7A shows representative flow cytometric analysis of day 4 total mesodermal populations for KDR and CD235a/b expression following induction with indicated BMP4 and Activin A concentrations in addition to 5 ng/ml bFGF from day 1-4.

FIG. 7B shows representative flow cytometric analysis of day 4 total mesodermal population for KDR and CD34 expression following BMP4 and Activin A induction in matched samples to ‘FIG. 7A.

FIGS. 8A-8B shows representative vasculature development from day 4 BMP4/Activin A grid (related to FIG. 1).

FIG. 8A shows representative flow cytometric analysis of CD34 and CD31 expression on day 8 populations generated from mesoderm induced with the indicated amounts of BMP4 and Activin A. Mesoderm populations were dissociated and replated at day 4 in venous endothelial specification conditions (10 ng/ml VEGF-A, 30 ng/ml bFGF).

FIG. 8B shows representative flow cytometric analysis of venous-like (CD34⁺ CD31⁺ CD73⁺ CD184⁻) versus arterial-like (CD34⁺ CD31⁺ CD73⁺ CD184⁺) expression on day 8 populations generated from mesoderm induced with the indicated amounts of BMP4 and Activin A. Expression patterns are from the gated CD34⁺CD31⁺ gated fraction shown in FIG. 8A.

FIGS. 9A-9N shows individual values (related to FIG. 1).

FIGS. 9A-9E shows quantification of flow cytometric analysis of indicated populations in day 4 mesodermal populations induced with different concentrations of BMP4 and Activin A. Related to FIGS. 1D-1F, n=5-6.

FIG. 9F shows quantification of total cell number from day 4 mesodermal populations induced as indicated. Values are presented as fold relative to mesoderm induced with 3B0A. Related to FIG. 1C, n=3.

FIGS. 9G-9I shows quantification of RT-qPCR expression analysis of indicated genes in day 4 total mesoderm populations induced with different concentrations of BMP4 and Activin A. Related to FIG. 1G-11, n=3.

FIG. 9J shows the quantification of total number of day 8 cells generated from the different mesoderm populations. Values are presented as fold relative to the number generated from mesoderm induced with 3B0A. The different mesoderm populations were dissociated and the cells monolayer culture in media containing 10 ng/ml VEGF-A, 30 ng/ml bFGF for venous specification (black bars) or in the presence of 100 ng/ml VEGF-A, 30 ng/ml bFGF for arterial specification (red bar). Arterial cells were generated from 12B4A induced mesoderm. Related to FIGS. 1J-1M, n=3.

FIGS. 9K-9M shows quantification of flow cytometric analysis of day 8 total endothelial cells (CD34⁺ CD31⁺), venous-like endothelial cells (CD34⁺ CD31⁺ CD73⁺ CD184⁻), and arterial-like endothelial cells (CD34⁺ CD31⁺ CD73⁺ CD184⁺). Related to FIGS. 1L-1M, n=3.

FIG. 9N shows total day 8 venous EC yield from mesoderm generated with induction conditions. Values are presented as fold relative to the number generated from mesoderm induced with 3B0A. Total is calculated as day 8 venous-like EC CD34⁺ CD31⁺ CD73⁺ CD184⁻ frequency × day 8 total cells × day 4 KDR⁺ CD235a/b⁺ frequency × day 4 total cells. Related to FIG. 1M, n=3. Data are presented as mean ± SEM. For RT-qPCR analysis, expression values are normalized to levels of the housekeeping gene TBP.

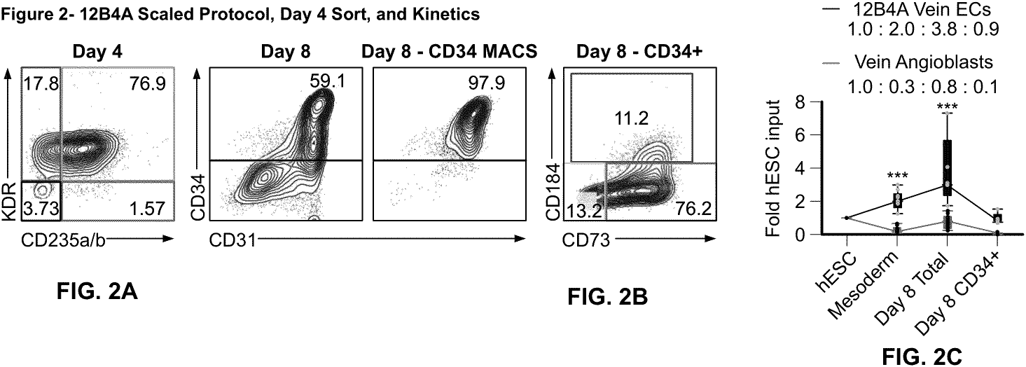

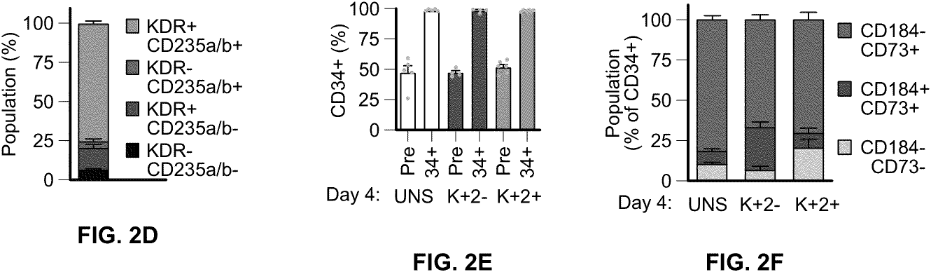

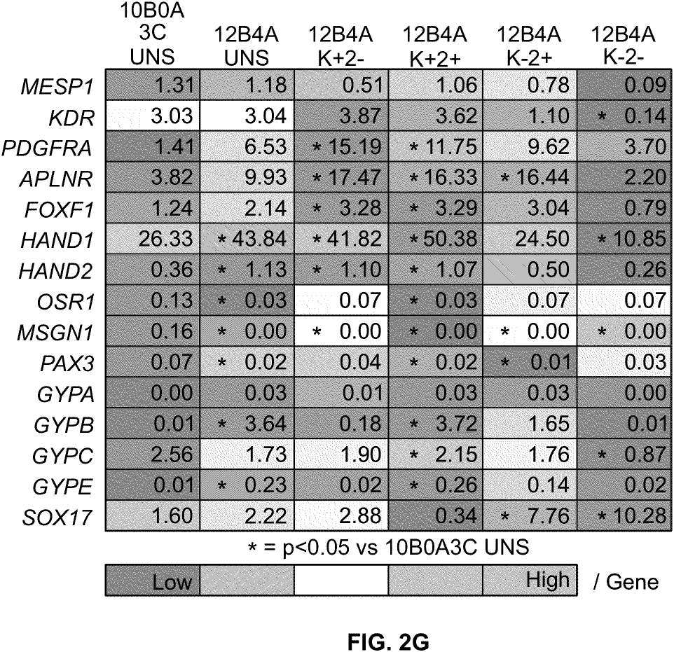

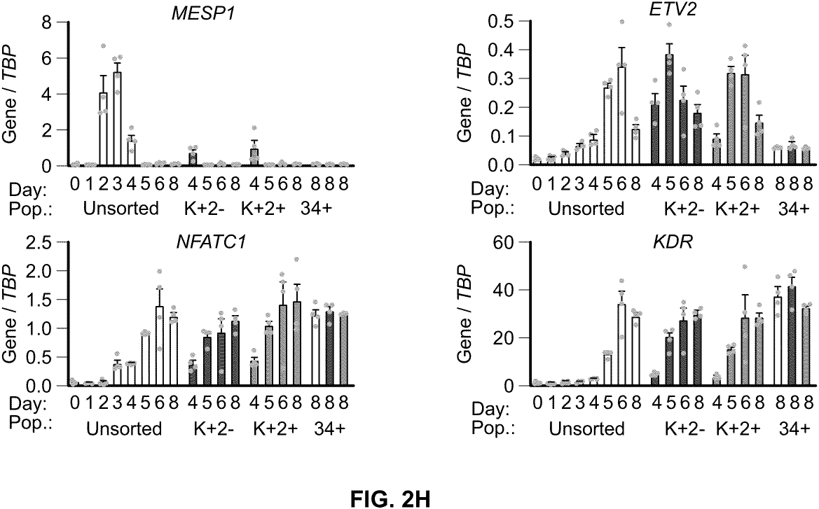

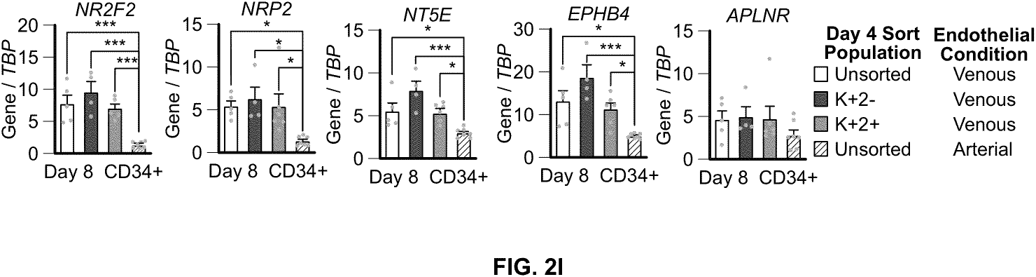

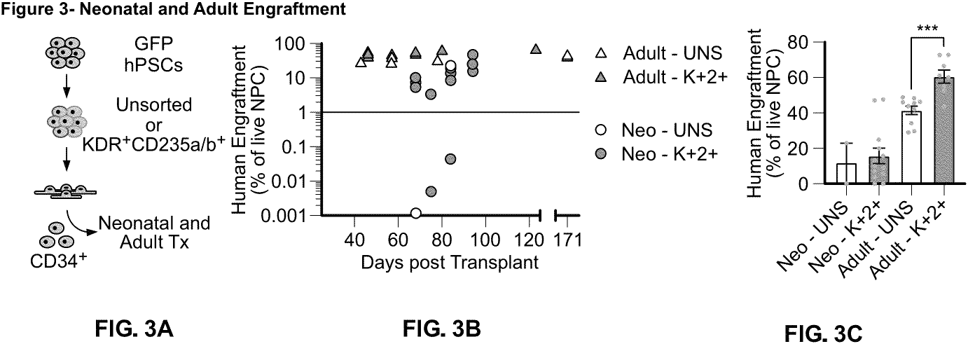

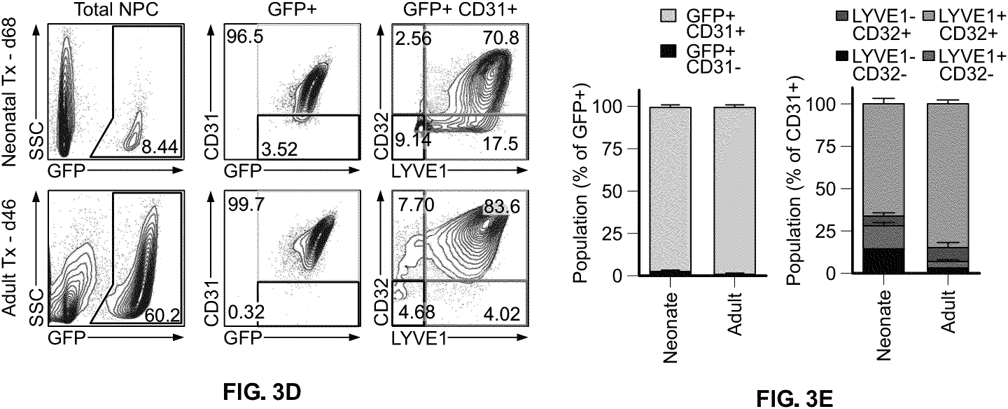

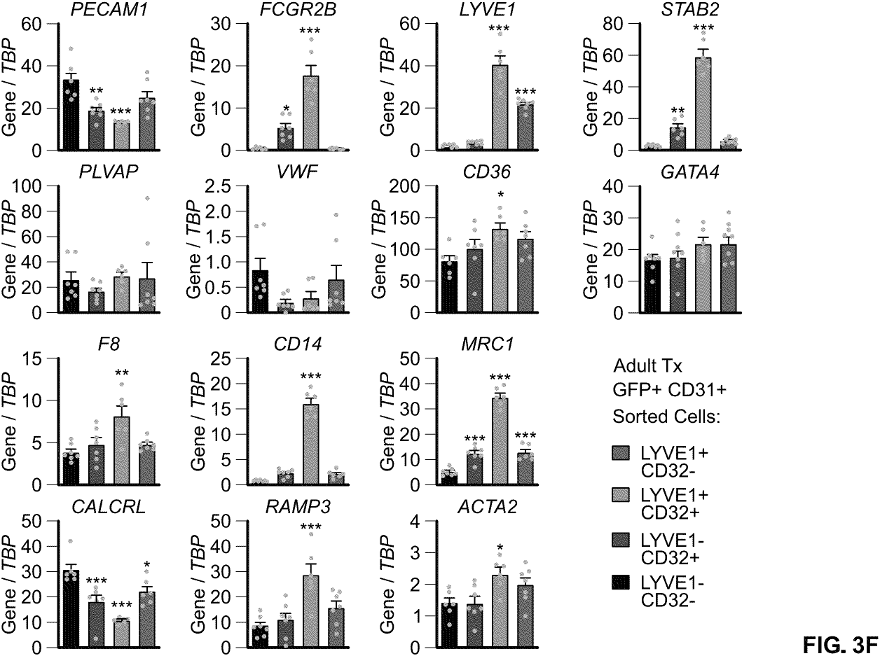

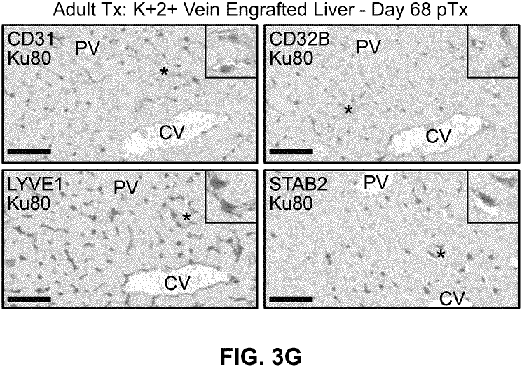

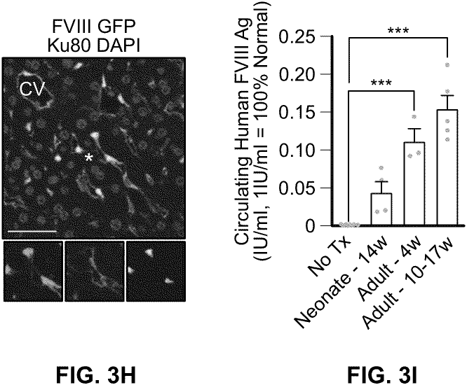

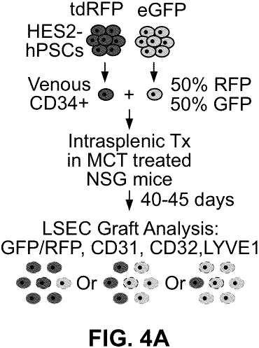

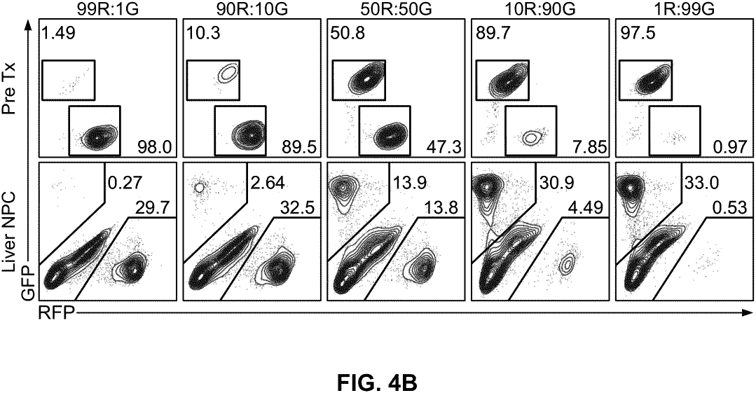

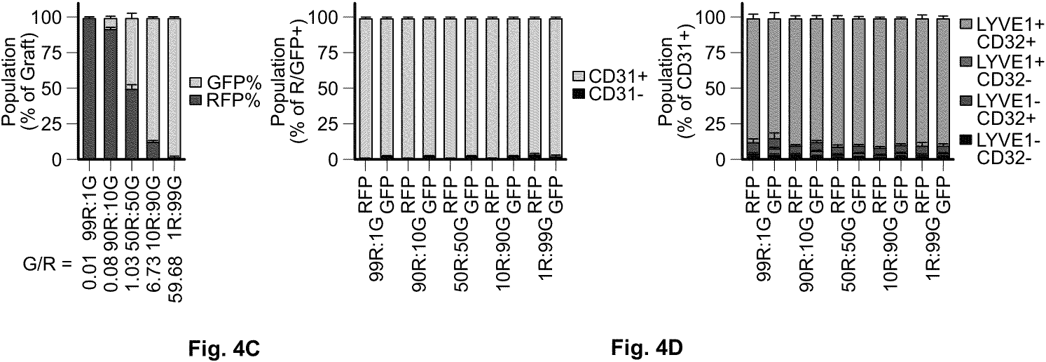

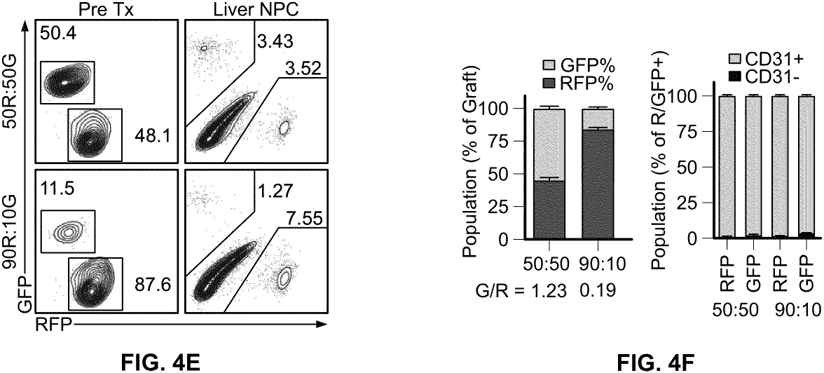

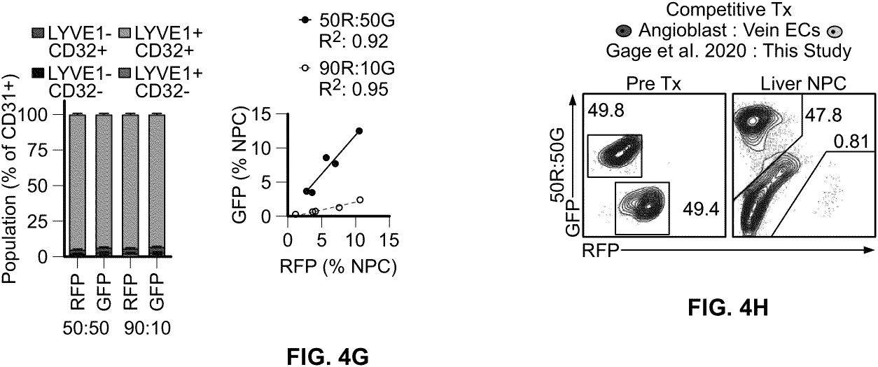

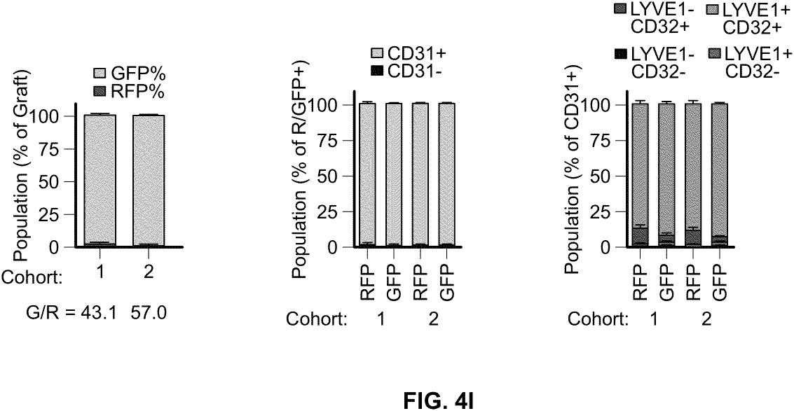

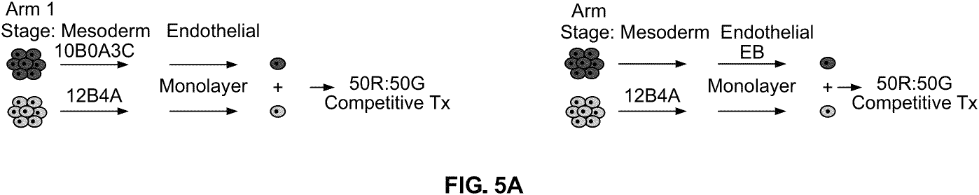

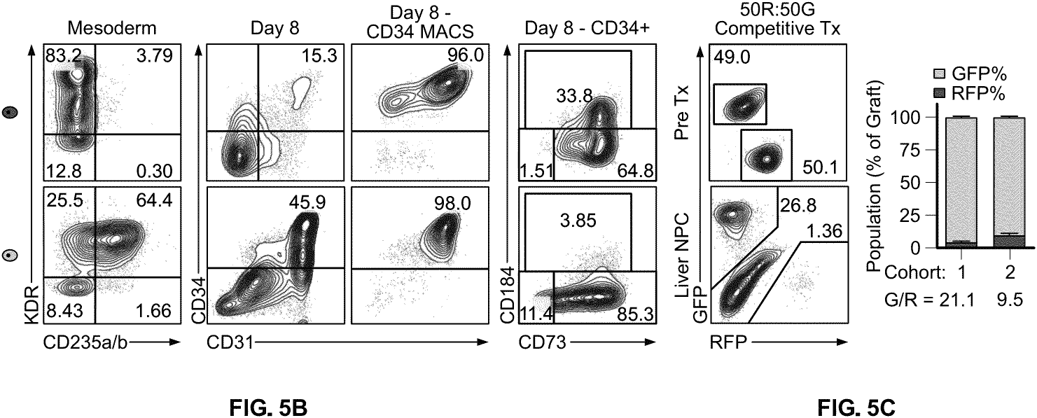

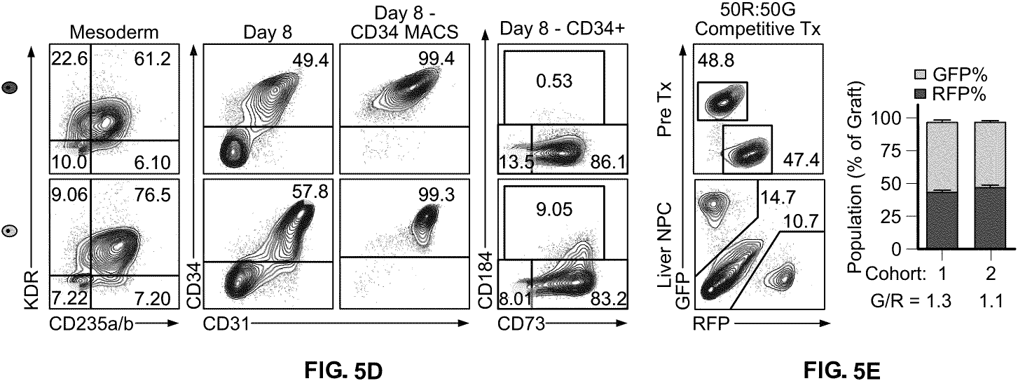

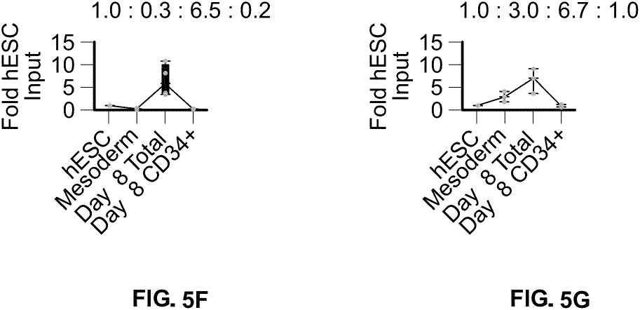

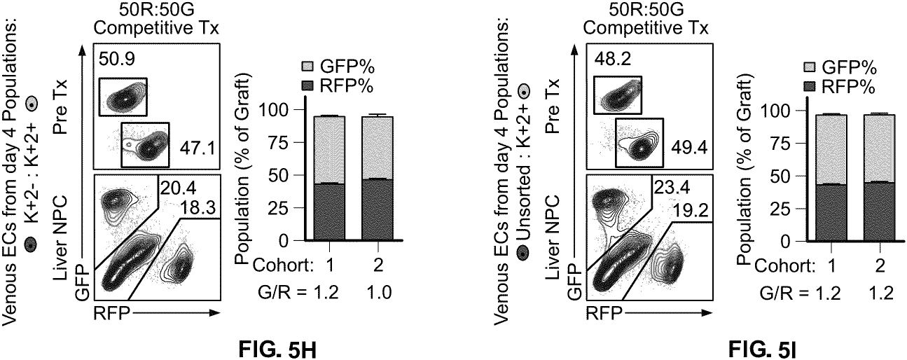

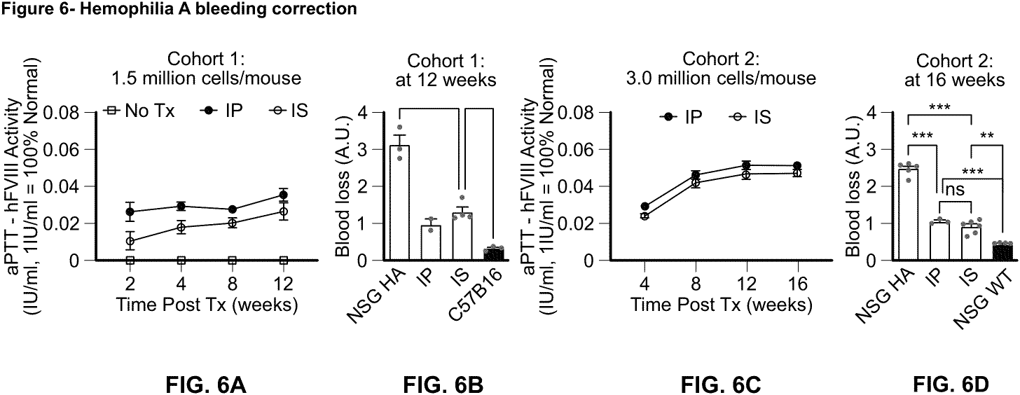

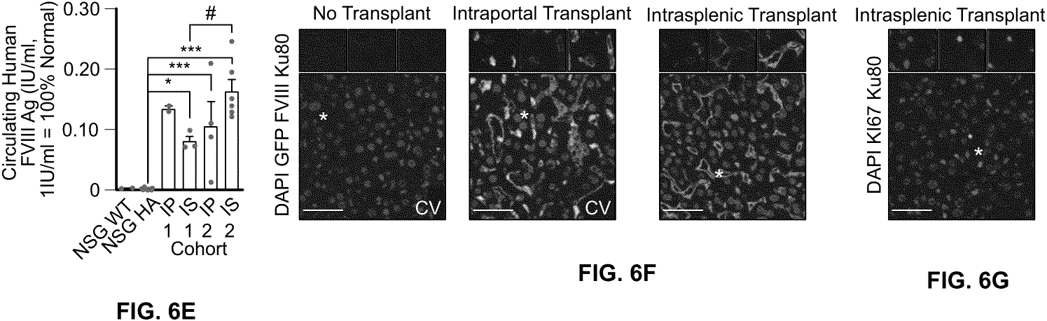

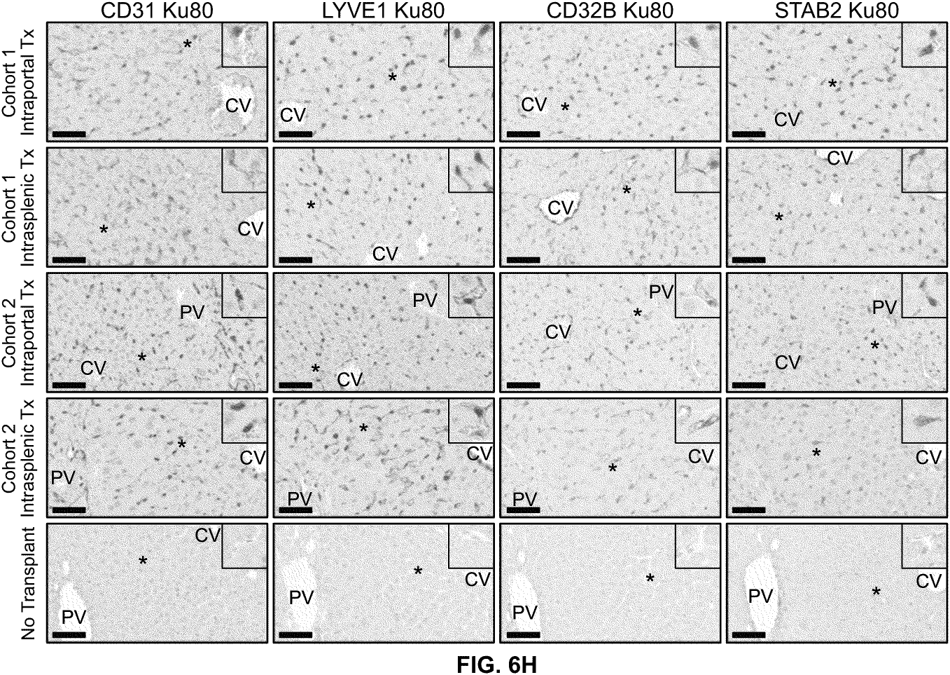

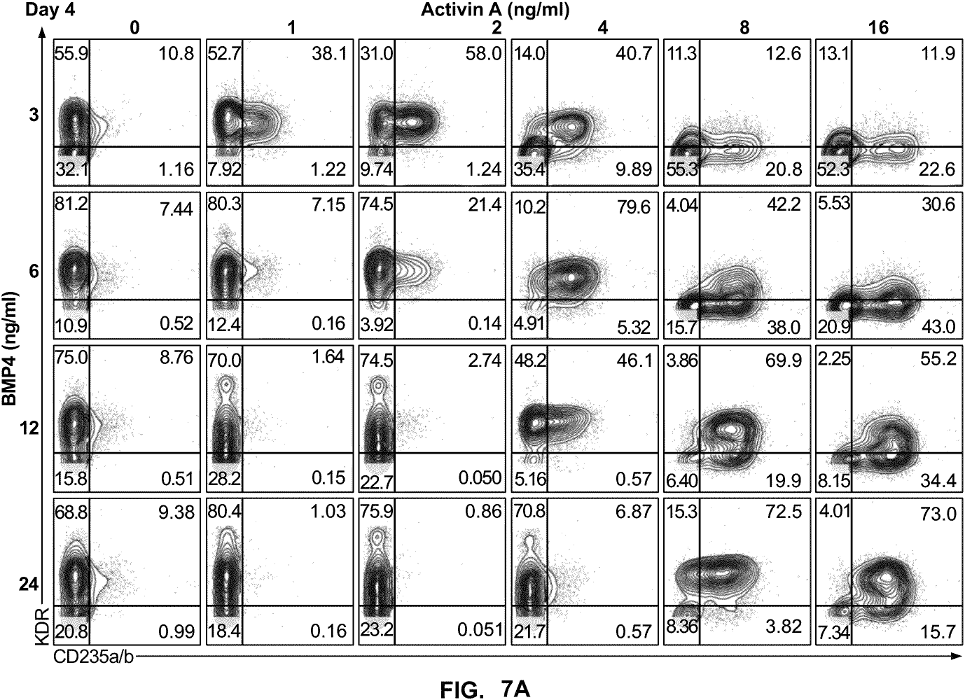

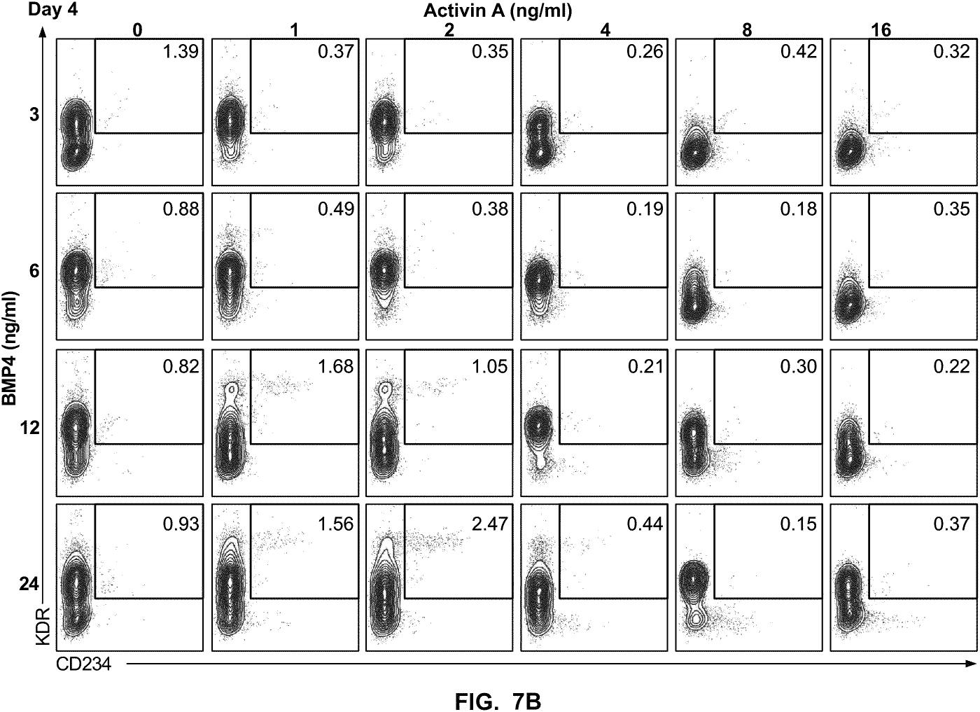

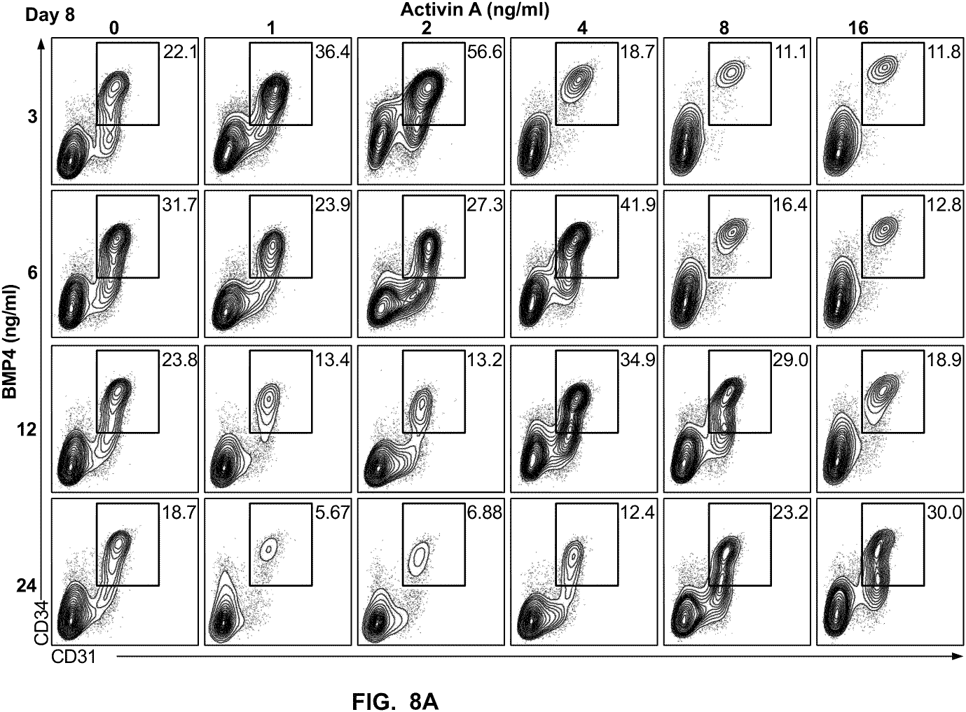

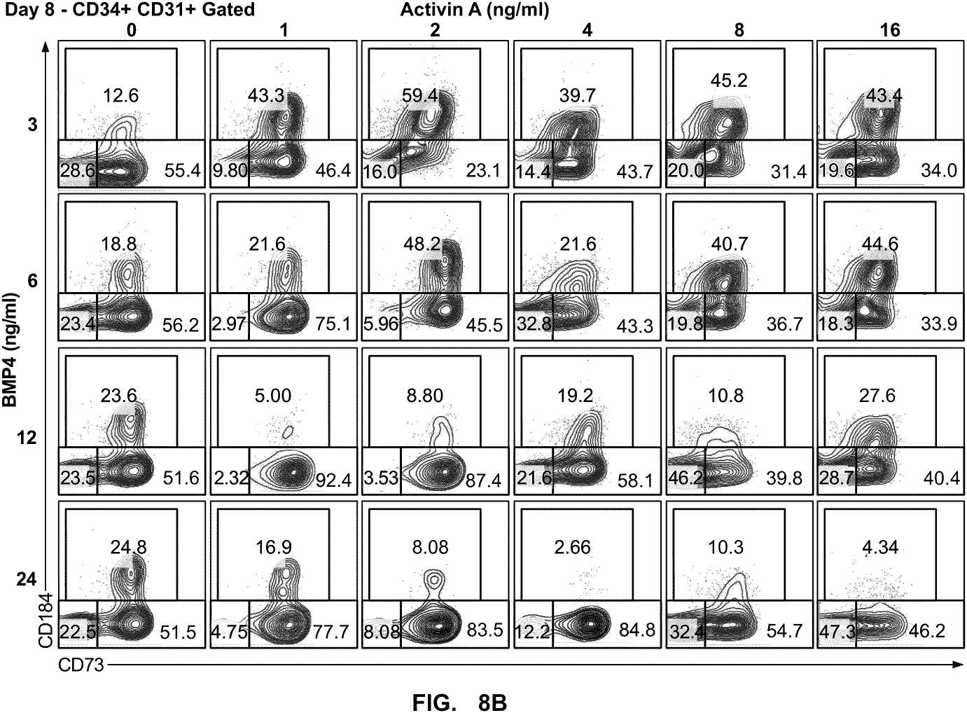

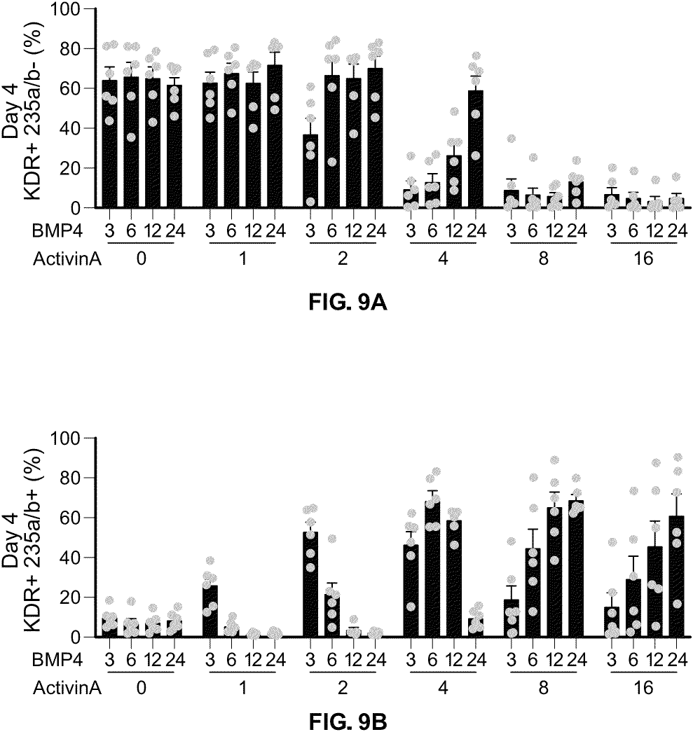

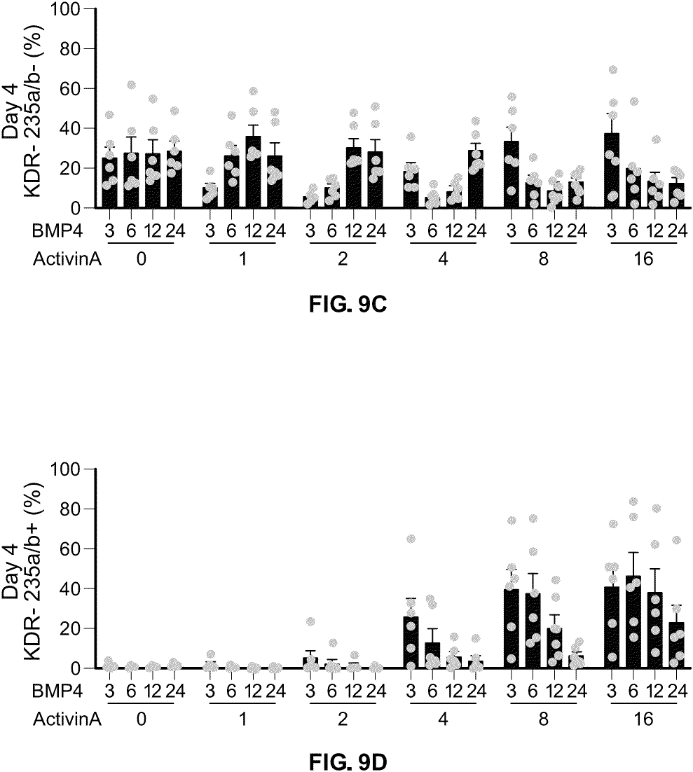

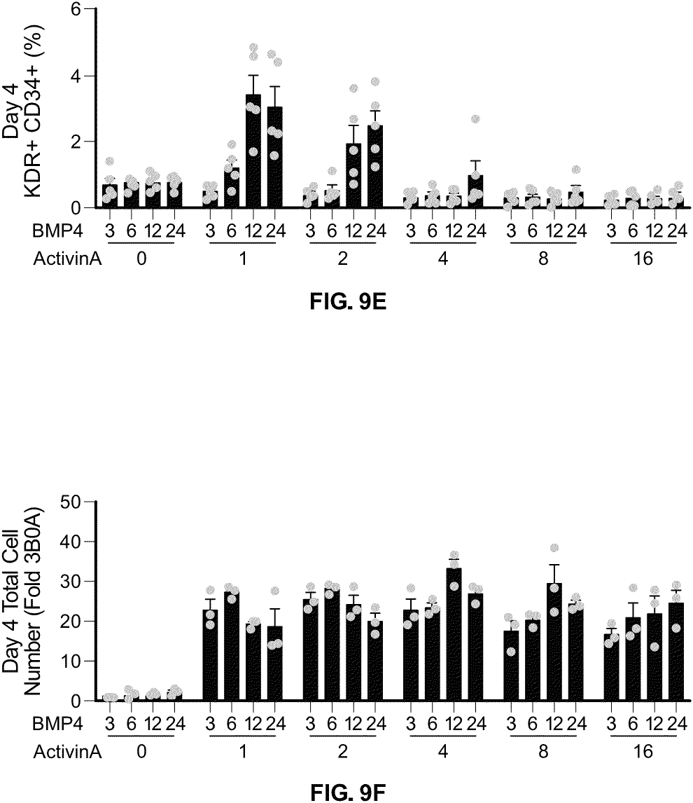

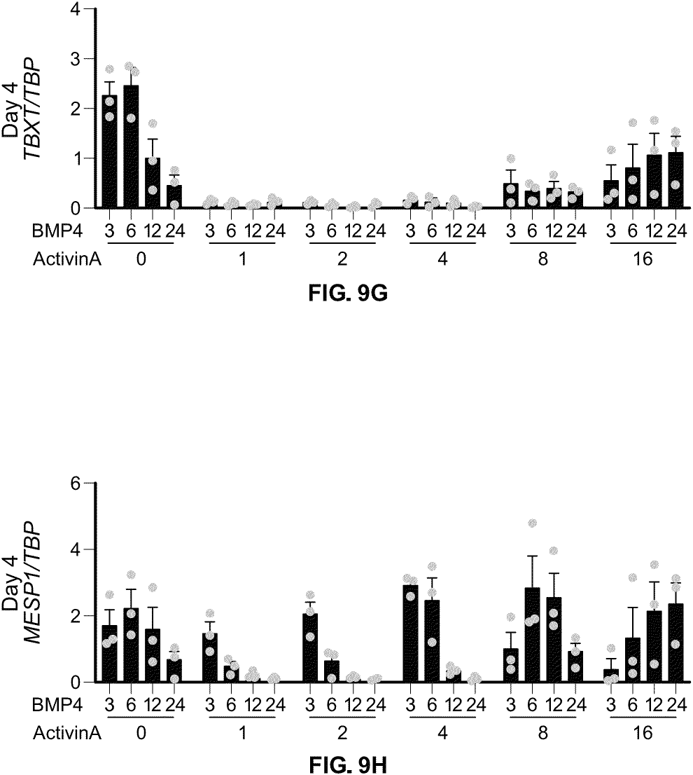

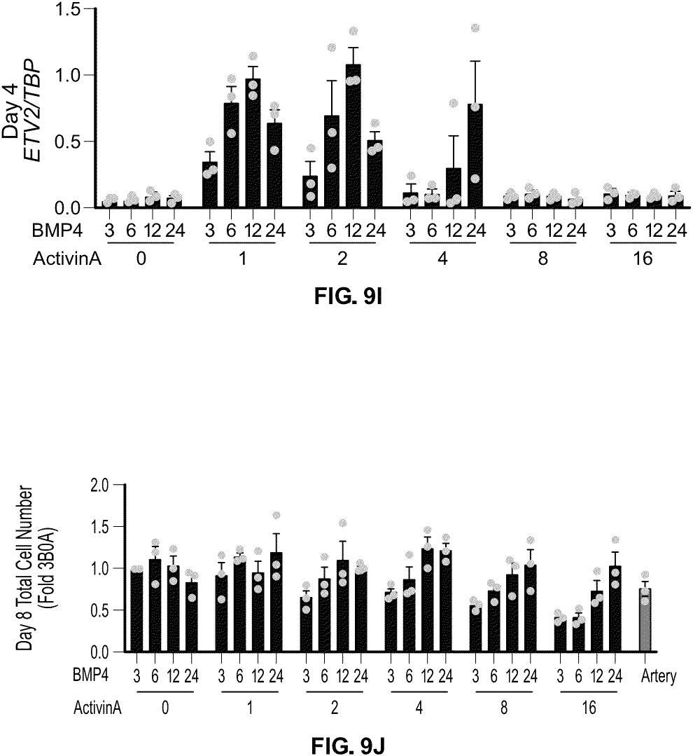

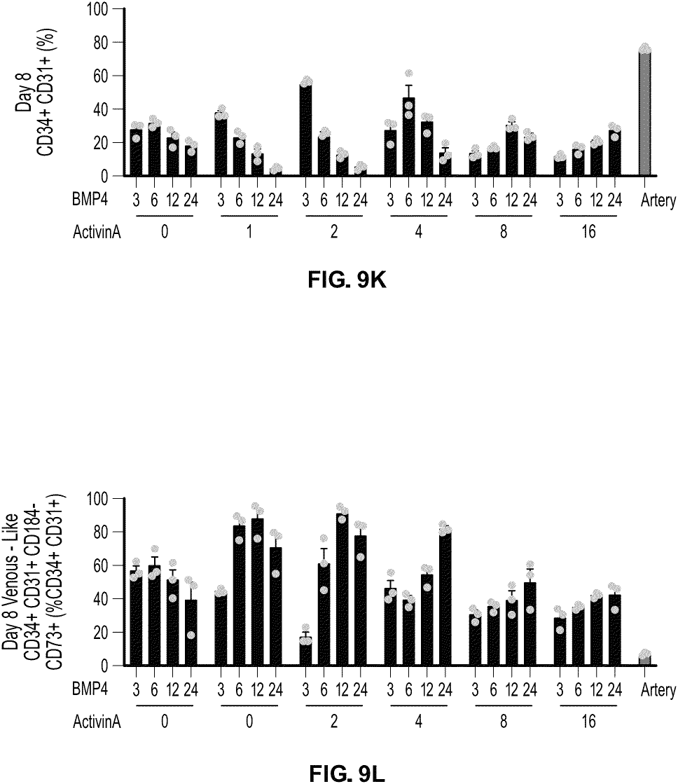

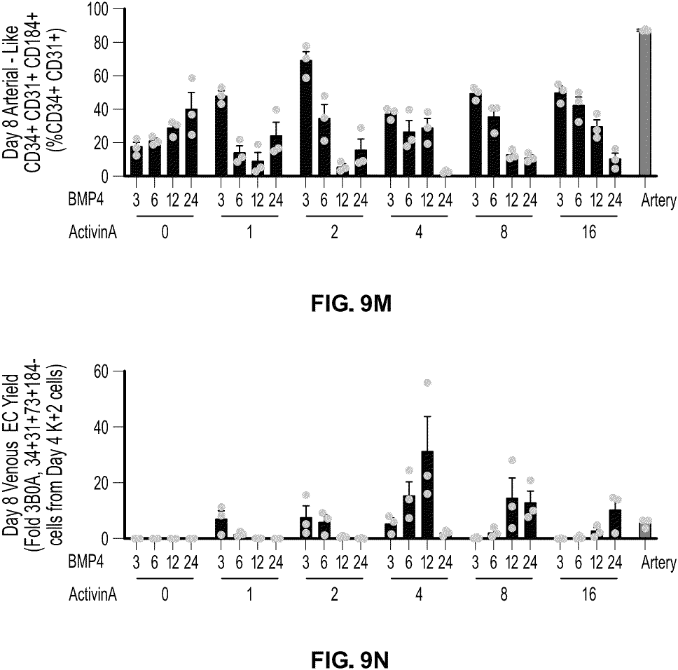

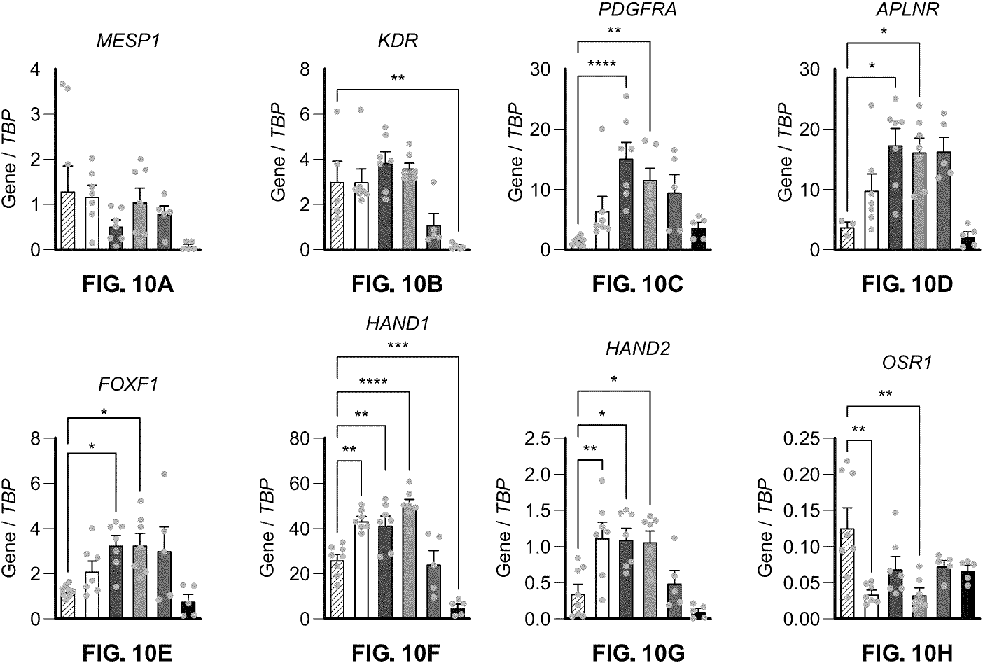

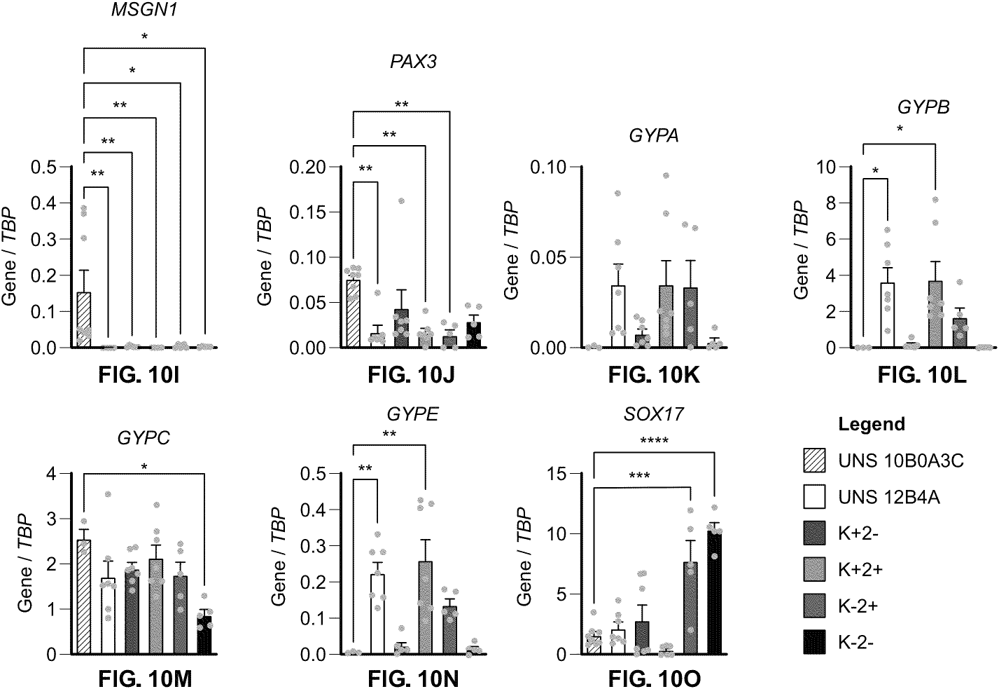

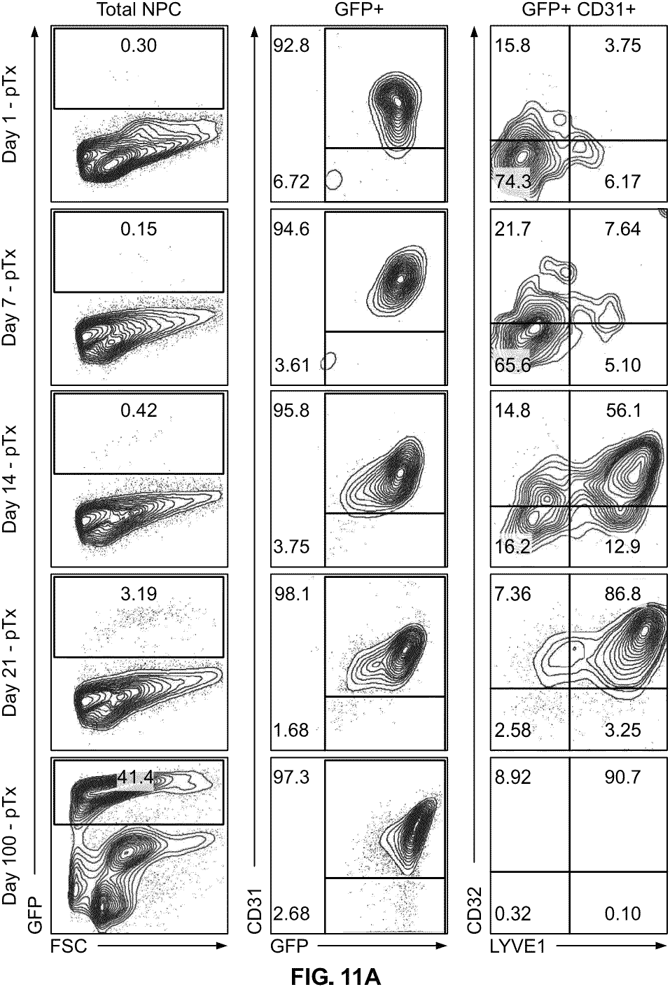

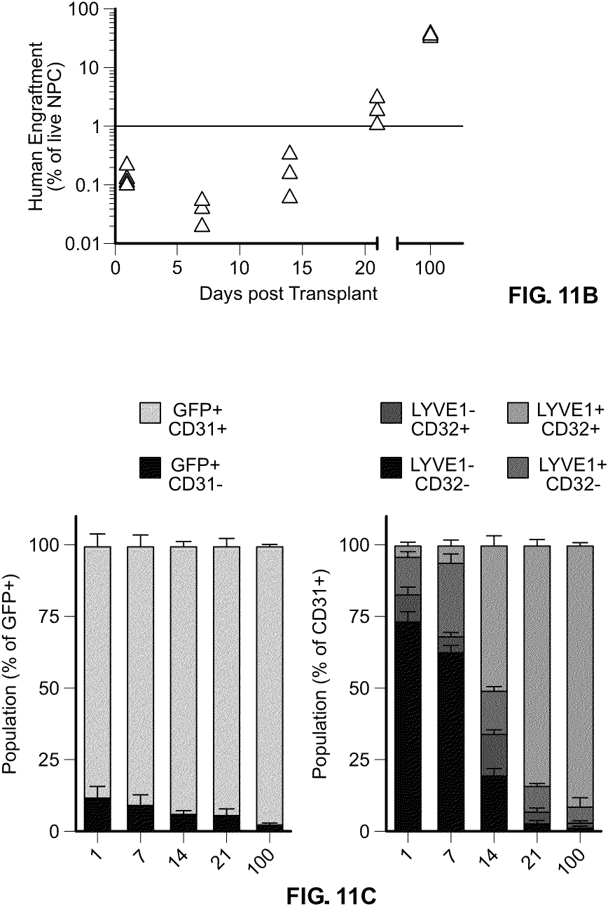

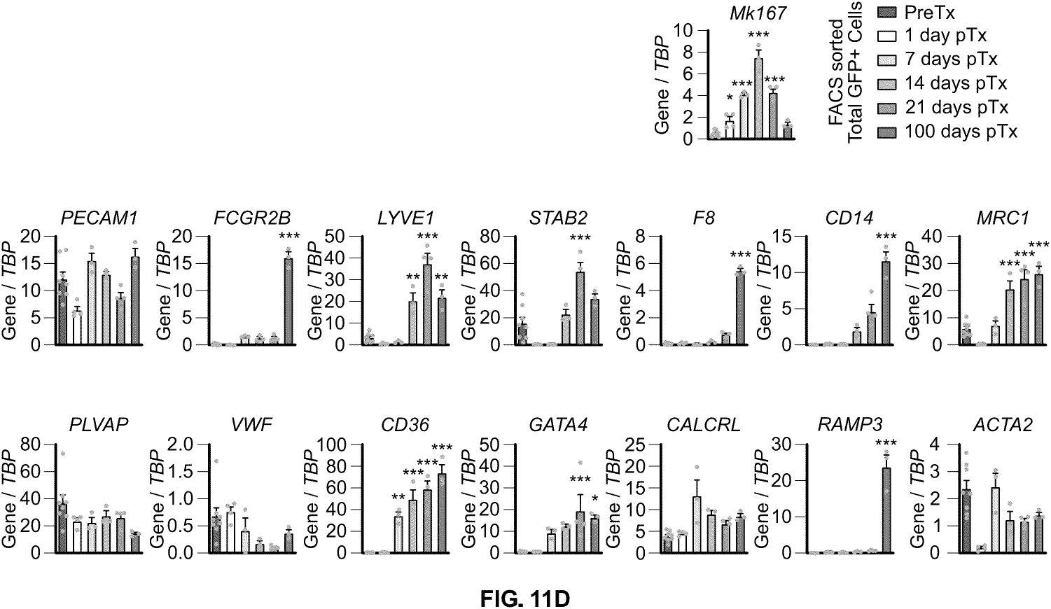

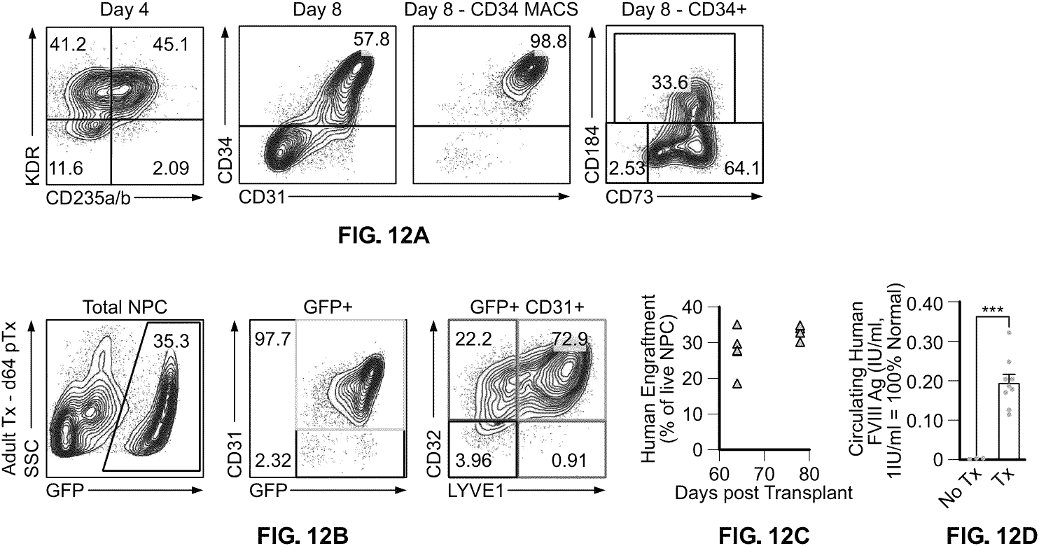

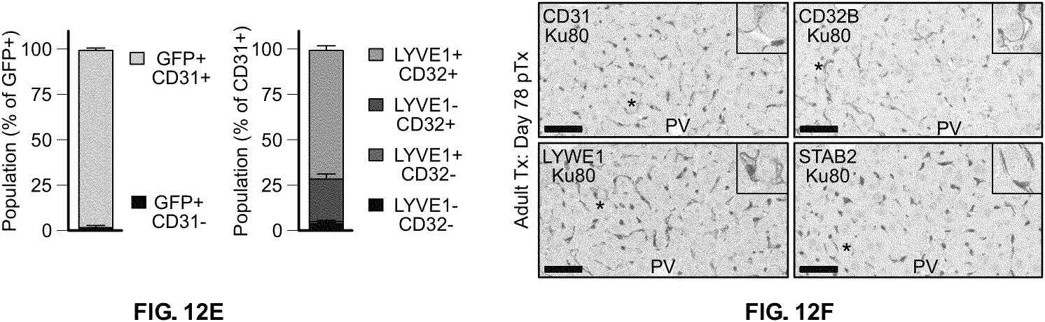

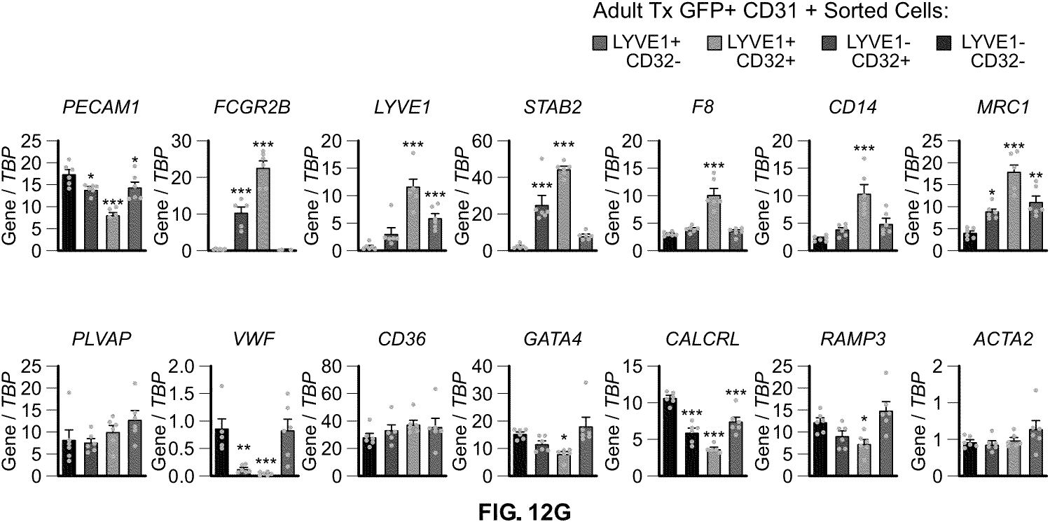

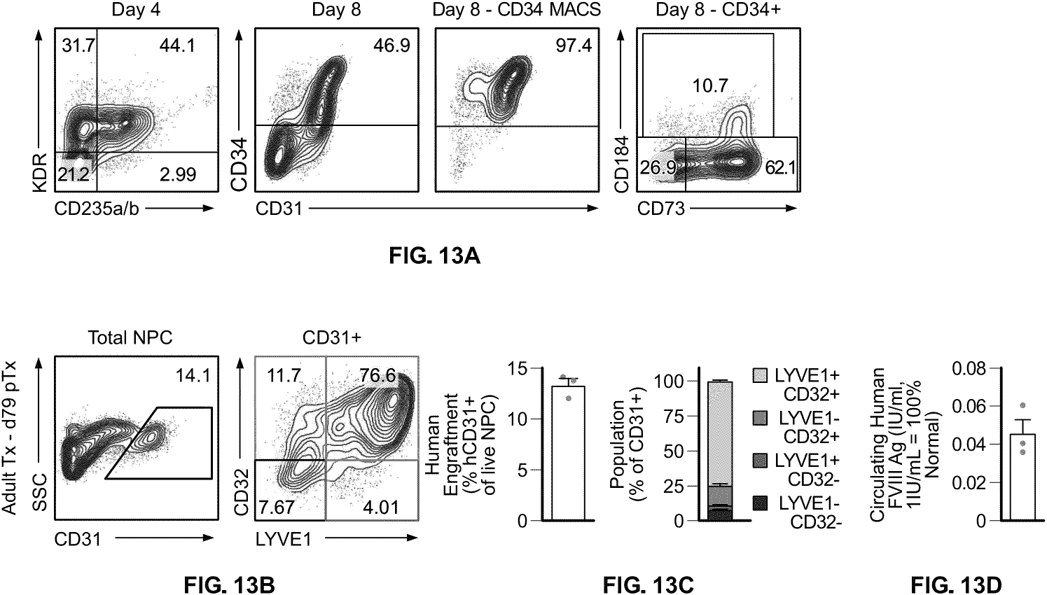

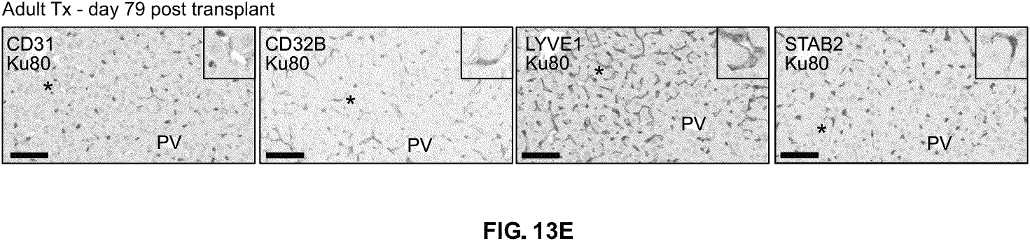

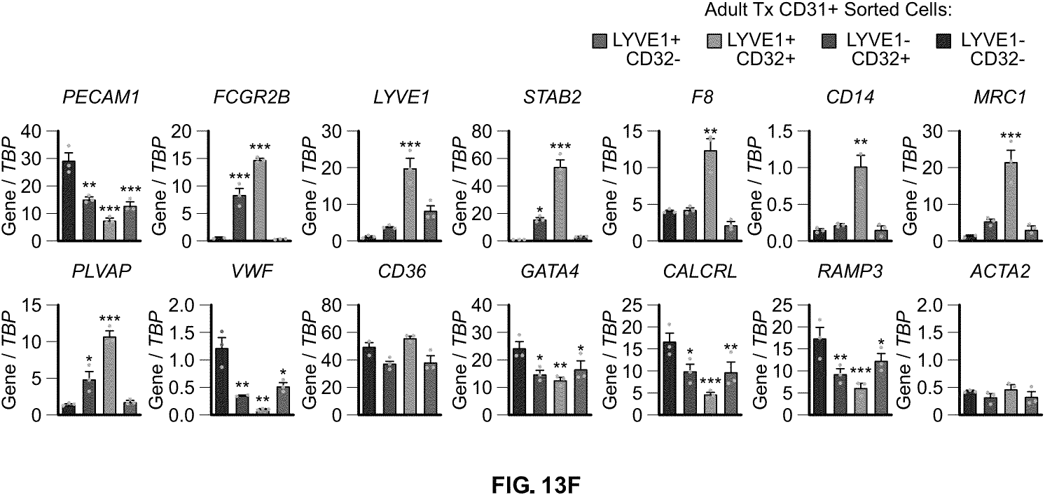

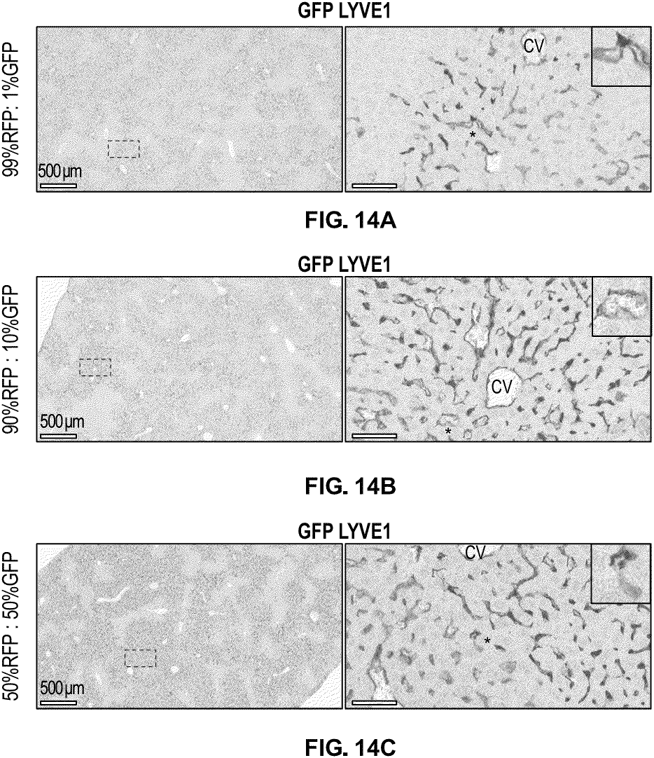

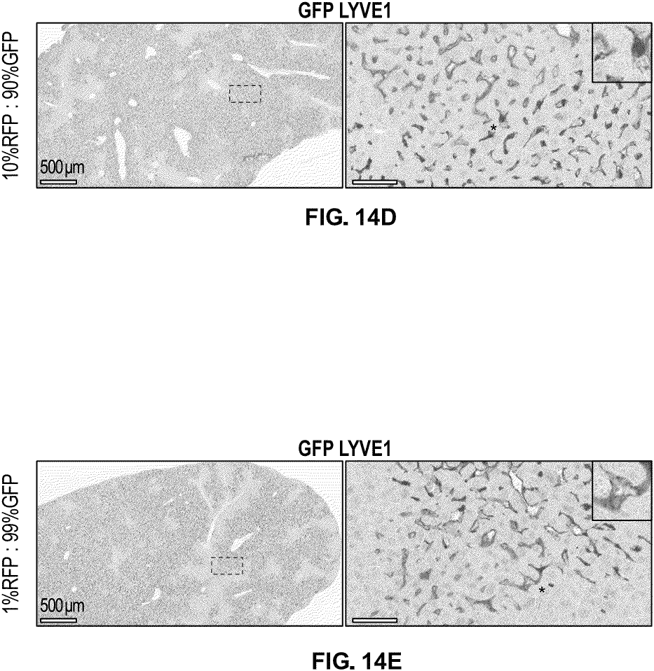

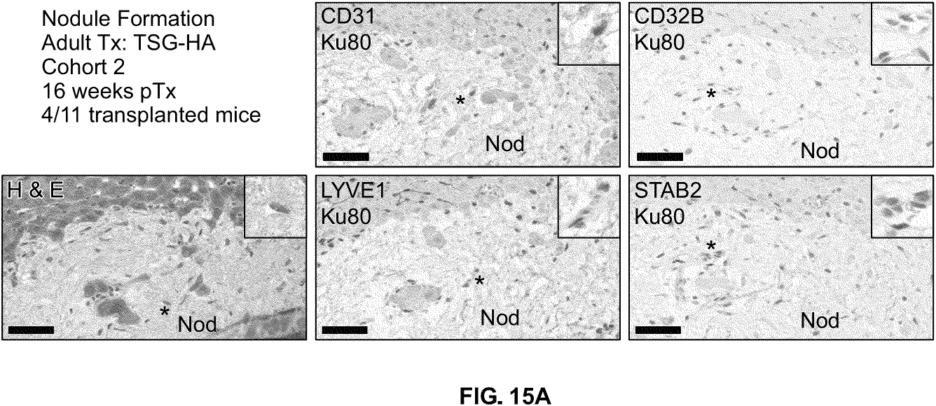

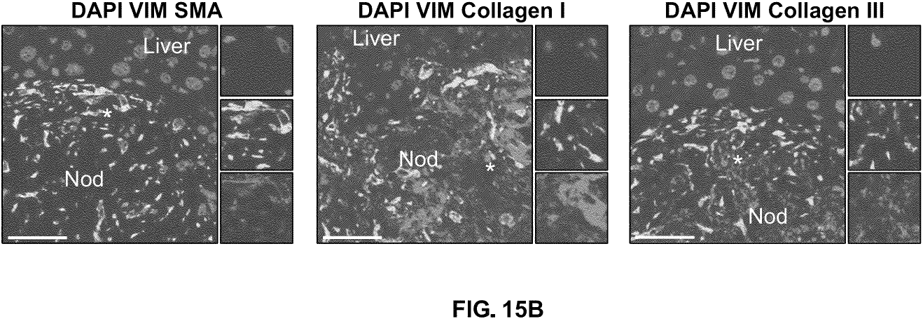

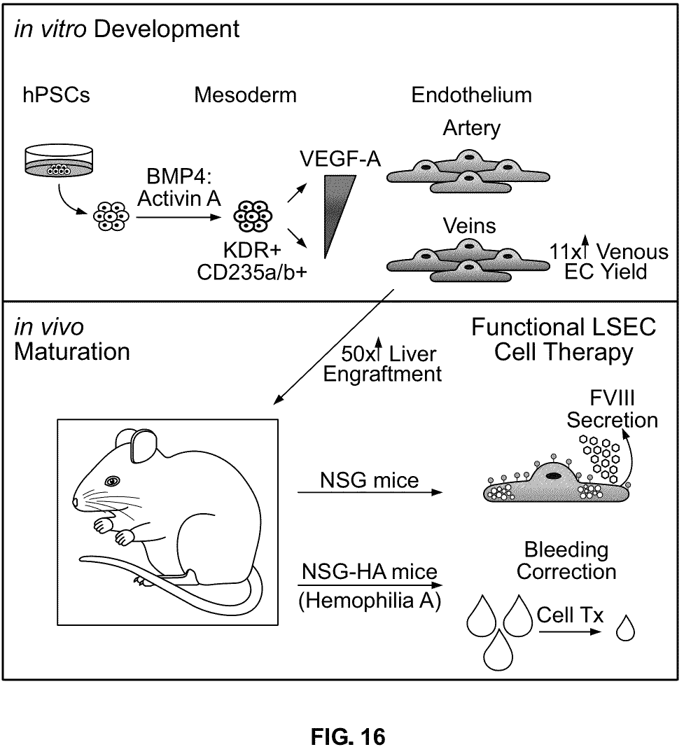

FIGS. 10A-10O shows individual values (related to FIG. 2).

FIGS. 10A-10O shows the quantification of RT-qPCR expression analysis of indicated genes in unsorted (UNS) or FACS sorted day 4 mesoderm populations. Data are presented as mean ± SEM. n=3-8 replicates per gene / sample with expression values normalized to levels of the housekeeping gene TBP. ANOVA with Bonferroni test, *p<0.05, **p<0.01 ***p<0.001 as indicated.

FIGS. 11A-11D shows the kinetics of LSEC engraftment (related to FIG. 3).

FIG. 11A shows representative flow cytometric analysis of GFP⁺ cells NPC engraftment from unsorted 12B0A-induced mesoderm-derived day 8 CD34+ VECs at indicated days post intrasplenic transplant in MCT treated adult NSG mice. hPSC-derived cells (GFP⁺), human endothelial engraftment (GFP⁺ CD31⁺) and human LSEC phenotype (GFP⁺ CD31⁺ CD32⁺ LYVE1⁺) are depicted.

FIG. 11B shows quantification of total GFP⁺ engraftment levels during early (day 1-21) and late (day 100) engraftment periods, n=3-5 per time point.

FIG. 11C shows quantification of GFP⁺ engrafting compositions at indicated days post-transplant, n=3-5 per time point.

FIG. 11D shows RT-qPCR analysis of proliferation (MKI67) and LSEC related markers in pre transplant day 8 12B4A-derived CD34⁺ VECs and FACS purified total GFP⁺ cells recovered after the indicated days post-transplant, n=3-8 per timepoint. (TBP normalized mean ± SEM, one-way ANOVA, *p<0.05, **p<0.01, ***p<0.001, vs pretransplant VECs).

FIGS. 12A-12G shows the generation and functional analyses of LSECs from H1-GFP hESCs.

FIG. 12A shows representative H1-GFP hESC derived flow cytometric analysis of day 4 mesoderm populations for KDR and CD235a/b expression following day 1-4 treatment with 12 ng/ml BMP4, 4 ng/ml Activin A, and 5 ng/ml bFGF in scaled suspension embryoid body culture. Day 8 pre and post CD34 targeted MACS enrichment for venous ECs assessed by flow cytometry for CD31, CD34, CD184, and CD73 indicating efficient VEC generation.

FIG. 12B shows representative flow cytometric analysis of NPC engraftment from H1-GFP day 8 VEC transplantation adult NSG transplant model generated from 12B4A mesoderm unsorted at day 4.

FIG. 12C shows quantification of human (GFP⁺) engraftment in NSG transplanted mice, n=9.

FIG. 12D shows plasma levels of human FVIII antigen in non-transplanted and H1-GFP VEC transplanted mice. (mean ± SEM, n=3-9, students T-test, ***p<0.001).

FIG. 12E shows quantification of flow cytometric analysis of GFP⁺ graft composition indicating the presence of endothelial cells (GFP⁺ CD31⁺) and LSECs (GFP⁺ CD31⁺ CD32⁺ LYVE1⁺). (adult Tx: n=9).

FIG. 12F shows histological analysis of H1-GFP derived grafts. Sections were analyzed with human specific endothelial/LSEC markers (CD31, LYVE1, CD32B, and STAB2) and the human specific nuclear antigen Ku80. “*” indicates location of enlarged inset panels; scale bar represents 60 µm.

FIG. 12G shows RT-qPCR analysis of LSEC markers in FACS isolated DAPIGFP⁺ CD31⁺ with variable CD32 and LYVE1 expression levels from H1-GFP derived day 8 venous EC transplantation in adult NSG mouse model. (TBP normalized mean ± SEM, one-way ANOVA, n=6, *p<0.05, **p<0.01, ***p<0.001, vs GFP⁺CD31⁺ cells).

FIGS. 13A-13F shows generation and functional analyses of LSECs from PGPC17-11 iPSCs.

FIG. 13A shows representative PGPC17-11 iPSC derived flow cytometric analysis of day 4 mesoderm populations for KDR and CD235a/b expression following day 1-4 treatment with 12 ng/ml BMP4, 4 ng/ml Activin A, and 5 ng/ml bFGF in scaled suspension embryoid body culture. Day 8 pre and post CD34 targeted MACS enrichment for VECs assessed by flow cytometry for CD31, CD34, CD184, and CD73 indicating efficient VEC generation.

FIG. 13B shows representative flow cytometric analysis of NPC engraftment from PGPC17-11 day 8 VEC transplantation adult NSG transplant model generated from 12B4A mesoderm unsorted at day 4 using human specific CD31 staining to determine iPSC derived graft size.

FIG. 13C shows quantification of flow cytometric analysis of human CD31⁺ graft composition indicating the presence LSECs (CD31⁺ CD32⁺ LYVE1⁺). (adult Tx: n=3).

FIG. 13D shows plasma levels of human FVIII antigen in PGPC17-11 venous EC transplanted mice at 79 days post-transplant, n=3.

FIG. 13E shows histological analysis of PGPC17-11 derived grafts. Sections were analyzed with human specific endothelial/LSEC markers (CD31, LYVE1, CD32B, and STAB2) and the human specific nuclear antigen Ku80. “*” indicates location of enlarged inset panels; scale bar represents 60 µm.

FIG. 13F shows RT-qPCR analysis of LSEC markers in FACS isolated DAPI⁻CD31⁺ with variable CD32 and LYVE1 expression levels from PGPC17-11 derived day 8 VEC transplantation in adult NSG mouse model. (TBP normalized mean ± SEM, one-way ANOVA, n=3, *p<0.05, **p<0.01, ***p<0.001, vs CD31⁺ CD32⁻ LYVE1-cells).

FIGS. 14A-14E shows competitive transplantation histology (related to FIG. 4).

FIGS. 14A-14E shows histological analysis of competitive transplantation validation experiments described in FIG. 4B using matched GFP and RFP versions of 12B4A-induced mesoderm-derived VECs. Total LSEC engraftment (LYVE1, pink, both GFP and RFP lines) and GFP (DAB-brown, GFP line only) reveals spatial arrangement of competitive liver vascular engraftment from indicated RFP:GFP ratio inoculums at day 42-43 post-transplant. Note that brown DAB deposition partially obscures pink LYVE1 staining in GFP⁺LYVE1⁺ LSECs. Images are depicted at low magnification (left) and high magnification (right) with dashed black rectangle indicating high magnification region. “*” indicates location of enlarged inset panels in high magnification image; scale bar represents 500 µm in left image and 60 µm in right image.

FIGS. 15A-15B shows incidental histological findings (related to FIG. 6). During the course of histological analysis of NSG-HA mice receiving VEC therapy as in FIG. 6, histological observations were made specifically in Cohort 2 at 16 weeks post-transplant with mice having received a high cell dose (3 million cells per mouse). These observations were not seen in NSG-HA mice receiving no cells, in Cohort 1, or in FVIII competent control NSG mice used for these studies.

FIG. 15A shows liver nodules (Nod). In 4 of 11 mice, rare (1-5 per liver), small (0.5-2 mm) nodules were macroscopically observed in the liver of cell-treated mice. Histologically, nodules occurred in well-engrafted livers and were surrounded by hESC-derived LSECs. Within nodules, human (Ku80⁺) non-endothelial/LSEC cells were observed that were negative for CD31, CD32B, LYVE1, and STAB2; scale bar represents 60 µm.

FIG. 15B shows that these same nodules contained human derived vimentin and smooth muscle actin expressing cells with adjacent collagen I and collagen III deposition. These nodules are similar to those reported previously in neonatal transplants, although smaller and less frequent. “*” indicates location of enlarged inset panels; scale bar represents 50 µm in FIG. 15B.

FIG. 16 is a schematic overview of the present disclosure.

### DETAILED DESCRIPTION

Liver sinusoidal endothelial cells (LSECs) form the predominant microvasculature in the liver where they carry out many functions including the secretion of coagulation factor VIII (FVIII). To investigate the early origins of human LSECs, an efficient and scalable protocol was developed to produce human pluripotent stem cell (hPSC)-derived venous endothelium from different mesoderm subpopulations. Using a sensitive and quantitative vascular competitive transplantation assay, it was demonstrated that venous endothelial cell (VEC) populations generated from BMP4-induced and Activin A-induced mesoderm characterized by KDR⁺ CD235a/b⁺ expression was 50-fold more efficient at engrafting the LSEC compartment in the liver of NSG mice than venous populations generated from KDR⁺ CD235a/b⁻mesoderm induced by BMP4 and WNT agonism.

When transplanted into immunocompromised Hemophilia A mice (NSG-HA), these VECs engrafted the liver, proliferated, and generated functional LSECs that secreted bioactive FVIII capable of correcting the bleeding phenotype. Together, these findings highlight the importance of appropriate mesoderm induction for the generation of specific cell types from hPSCs and demonstrate that, with this approach, it is possible to generate functional LSECs in a pre-clinical model of Hemophilia A.

Thus, this disclosure describes methods of making mesodermal cell-derived VECs. As described herein, such mesodermal cell-derived VECs subsequently can be used to produce LSECs in vitro or in vivo, which have a number of therapeutic applications.

Human pluripotent stem cells (hPSCs) can be used in the methods described herein. Human pluripotent stem cells (hPSCs) include, without limitation, human embryonic stem cells (hESCs), human induced pluripotent stem cells (iPSCs) or other human cells having a pluripotent phenotype.

The hPSCs can be contacted with at least one modulator of BMP signaling and at least one modulator of Activin signaling in order to generate the mesodermal cells described herein (i.e., KDR+, CD235a/b+). Modulators of BMP signaling and modulators of Activin signaling are known in the art; representative modulators of BMP signaling include, without limitation, BMP4, BMP2, or small molecule BMP signaling agonists (e.g., ventromorphins [PMID 28787124], ID1 or ID2 [PMID 23527084], chromenone 1 [PMID 35108017], SB 4 [CAS number 100874-08-6] or similar), while representative modulators of Activin signaling include, without limitation, Activin A, NODAL, or small molecule TGFbeta signaling agonists.

As described herein, under appropriate conditions, mesoderm cells having a phenotype of KDR+ and CD235a/b+ are produced. The mesodermal KDR+ CD235a/b+ cells also can be phenotypically PDGFRa+, CD56+ and APLNR+.

Also as described herein, under appropriate conditions, the mesodermal KDR+ CD235a/b+ cells can be induced to differentiate into VECs (i.e., “mesodermal cell-derived VECs”), which typically have a phenotype of CD34+, CD31+, CD73+ and CD184-. The mesodermal cell-derived VECs also can be phenotypically NR2F2+, NRP2+, NT5E+ and EPHB4+. Any number of compounds can be used to induce the mesodermal KDR+ CD235a/b+ cells into mesodermal cell-derived VECs. For example, mesodermal cell-derived VECs cells can be induced using VEGF-A, VEGF-B, VEGF-C, VEGF-D, PIGF, bFGF/FGF2, or small molecule signaling agonists of VEGFA or FGF.

It would be appreciated that the contacting step and the inducing step can be performed in culture (e.g., monolayer, adherent) or in embryoid bodies (EBs). Culture conditions for maintaining or differentiating the cells are described herein, and embryoid bodies are known in the art as aggregates of pluripotent stem cells or derivative differentiating cells.

Significantly, the mesodermal cell-derived VECs described herein can be differentiated into liver sinusoidal endothelial cell-like cells (LSEC-LCs), which are phenotypically CD31+, CD32+ and LYVE1+. The LSEC-LCs also can be phenotypically CD32B+, STAB2+ and FVIII+. The differentiation into LSEC-LCs can take place in vitro (e.g., in culture), however, as described herein, the mesodermal cell-derived VECs differentiate extremely efficiently into functional LSECs in vivo. For example, the mesodermal cells described herein (i.e., KDR+ CD235a/b+) are capable of generating a population of VECs that exhibits at least a 20-fold greater (e.g., at least a 30-fold, 40-fold, 50-fold greater) engraftment potential than venous angioblast cells derived from mesoderm cells having a phenotype of KDR+ and CD235a/b-

Any number of compounds can be used to induce the mesodermal cell-derived VECs into LSEC-LCs in vitro. For example, mesodermal cell-derived VECs cells can be induced to form LSEC-LCs in the presence of bFGF, TGFbeta inhibition, cAMP agonism, and an agonist of BMP9 signaling. Alternatively, mesodermal cell-derived VECs can be induced to differentiate into LSEC-LCs using hypoxic conditions.

The LSECs described herein (i.e., derived from the engraftment of mesodermal cell-derived VECs) can be characterized by greater levels of expression of FCGR2B, LYVE1, STAB2, F8, CD14, MRC1, CD36 and RAMP3 and lesser levels of expression of PECAM1, VWF and CALCRL relative to VEC-derived non-LSEC endothelium (i.e., having a phenotype of CD31+ CD32- LYVE1-).

The cells described herein, particularly the mesodermal cell-derived VECs, can be used to treat a subject suffering from a liver disease. For example, the cells described herein can be administered or delivered to the subject, resulting in efficient engraftment in the liver. As used herein, treating refers to an approach intended to obtain a beneficial or desired result, which may include alleviation of symptoms, or delaying or ameliorating a disease progression. It would be appreciated that cells can be administered to a subject via intravenous delivery (e.g., infusion via the hepatic portal vein or systemic circulation). In some instances, it may be desirable to administer or deliver cells more than once to a subject (e.g., multiple times, a plurality of times).

As demonstrated herein, engrafted mesodermal cell-derived VECs efficiently differentiate into LSECs and are able to produce therapeutically effective amounts of the Factor VIII (FVIII) protein (e.g., sufficient levels to reverse the severe bleeding phenotype in a subject suffering from Hemophilia A), thereby treating Hemophilia A. Given the number of cells that engraft and differentiate, however, any number of other monogenic endothelial diseases can be treated in addition to other liver diseases such as, without limitation, acute drug liver injury (e.g., Sinusoidal Obstruction Syndrome (e.g., human monocrotaline toxicity among other agents), or Acetaminophen overdose), chronic liver injury (e.g., NASH, NAFLD, Cirrhosis, chronic drug injury), liver cancer (e.g., primary, hepatocellular carcinoma; or secondary (e.g., colon, breast, pancreatic) liver metastatic cancer).

In some instances, it may be desirable to render the hPSCs, the mesoderm cells, the mesodermal cell-derived VECs or the LSECs hypoimmunogenic. Cells can be rendered hypoimmunogenic by genetic addition or removal of immunoregulatory antigens, thereby resulting in cell populations that are not readily rejected by the recipient despite incomplete immunological matching.

Similarly, in some instances, it may be desirable to genetically engineer the hPSCs, the mesoderm cells, the mesodermal cell-derived VECs or the LSECs. Any of such cells can be genetically engineered to express nucleic acid encoding one or more exogenous proteins (e.g., functional, structural and/or therapeutic).

In accordance with the present invention, there may be employed conventional molecular biology, microbiology, biochemical, and recombinant DNA techniques within the skill of the art. Such techniques are explained fully in the literature. The invention will be further described in the following examples, which do not limit the scope of the methods and compositions of matter described in the claims.

### EXAMPLES

### Example 1-Animals

All experiments were done in accordance with institutional guidelines approved by the University Health Network Animal care committee for studies utilizing NSG mice in Toronto Canada (NOD.Cg PrkdcscidI12rgtm1Wjl /SzJ; Jackson Laboratory, local Ontario Cancer Institute colony). Experiments involving previously described NSG-HA mice (Merlin et al., 2019, Blood Adv., 3:825-38; Zanolini et al., 2015, Haematologica, 100:881-92) were performed at the Università del Piemonte Orientale following institutional approval. All animal studies were performed in 5-15 week old male and female mice for adult studies and P2-3 NSG neonates. Animals were maintained under standard conditions (12-hour light/dark cycle) in ventilated sterile microisolation cages as previously described (Gage et al., 2020, Cell Stem Cell, 27:254-69).

### Example 2-Cell Lines

hPSC lines were maintained on gelatin (0.1% w/v in PBS) and growth factor reduced Matrigel (0.25%v/v, Corning) coated dishes with irradiated mouse embryonic fibroblasts in DMEM/F12 KnockOut serum based hPSC culture media as previously described (Gage et al., 2020, Cell Stem Cell, 27:254-69) with 10-20 ng/ml rhbFGF (R&D). All cell lines were authenticated by providence, fluorescent protein expression, and confirmed to be karyotypically normal and mycoplasma free within two passages of experimental use. HES2 hESCs (Reubinoff et al., 2000, Stem Cells Transl. Med., 9:686-96) (karyotype: 46XX) were previously targeted at the human ROSA locus to constitutively express tdRFP or eGFP (Irion et al., 2007, Nat. Biotechnol., 25:1477-82). H1-GFP hESCs (karyotype: 46XY) were a gift from R. Moon (University of Washington, Seattle, USA) (Davidson et al., 2012, PNAS USA, 109:4485-90). PGPC17-11 iPSCs (karyotype: 46XY) (Hildebrandt et al., 2019, Stem Cell Reports, 13:1126-41) obtained from J. Ellis (SickKids Research Institute, Toronto, Ontario, Canada) were adapted from culture in mTeSR1 (Stemcell Technologies) to culture with irradiated mouse embryonic fibroblasts. These iPSCs were maintained as above with the addition of ROCK inhibitor (Y-27623, 10 µM, TOCRIS) to cultures at the time of passage and EB formation.

### Example 3-Directed Differentiation of Venous Endothelial Cells

hESC differentiation (FIG. 1A) was performed in embryoids bodies (EBs, day 1-4) and monolayer culture (day 4-8) under hypoxic conditions (5% CO₂, 5%O₂, 90%N₂). Initially, 85-95% confluent HES2-RFP, HES2-GFP, H1-GFP, PGPC17-11 hPSCs were dissociated to single cells (3-4 mins, TrypLE, ThermoFisher) before reaggregation at 5×10⁵ cells/ml, 5 ml per 6 cm Petri dish (VWR) to form 75 µm diameter EBs after 18 hours of orbital rotation (MaxQ 2000 shaker, ThermoFisher, 60 RPM). EBs were formed in “base media” consisting of StemPro34 (25% v/v, ThermoFisher), IMDM (75% v/v, ThermoFisher), ITS-X (1:10,000, ThermoFisher), penicillin/streptomycin (1%, ThermoFisher), L-glutamine (2 mM, ThermoFisher), ascorbic acid (50 µg/ml, SigmaAldrich), transferrin (150 µg/ml, ROCHE), and monothioglycerol (50 µg/ml, Sigma-Aldrich), supplemented for reaggregation with ROCK inhibitor (Y-27623, 10 µM, TOCRIS) and rhBMP4 (1 ng/ml, R&D). Eighteen to twenty hours later on day 1, EBs were transferred to 6-well or 10 cm PolyHema (5% w/v in 95% ethanol, Sigma-Aldrich, dried overnight) coated plates in primitive streak/mesoderm induction medias. During mesoderm optimization (FIG. 1) experiments at 1.25×10⁶ day 0 cells per well in 6-well plates, these day 1 medias consisted of “base media” supplemented with rhbFGF (5 ng/ml), BMP4 (3-24 ng/ml), and Activin A (0-16 ng/ml, R&D). During scaled-up optimized conditions (FIG. 2) 5×10⁶ day 0 cells as EBs were replated in 12 ml of “base media” supplemented with rhbFGF (5 ng/ml), BMP4 (12 ng/ml), and Activin A (4 ng/ml) per 10 cm plate.

On day 4, mesoderm specified EBs were dissociated (Trypsin-EDTA, Corning, 5 min) stopped with 50% v/v fetal calf serum (FCS, Wiscent) / IMDM, counted and replated on GFR-Matrigel coated (2.5% v/v in IMDM) 6-well or 10 cm plates to specify venous endothelium. 1×10⁶ cells / 6-well or 6×10⁶ cells / 10-cm plate were plated in “base media” supplemented with rhbFGF (30 ng/ml) and VEGF-A (10 ng/ml, R&D). On day 6, media was removed and replaced with freshly prepared day 4 media. To specify arterial lineage cells, day 4-8 media consisted of “base media” supplemented with rhbFGF (30 ng/ml) and VEGF-A (100 ng/ml). On day 8, endothelial cells were isolated by CD34 targeted MACS before cryopreservation (Cryostore CS10, STEMCELL Technologies) for future studies. Monolayer cultures were washed (PBS -Mg-Ca), dissociated (TrypLE, 10 mins, 37° C.), and stained for CD34 MACS (Miltenyi, 130-146-702) using 10 µl antibody / 5×10⁶ cells / 100 µl in base media supplemented with DNASE (1 U/ml, Millipore) for 30 minutes at 4° C. before purification over two columns in series (either MS or LS depending on cell number) to isolate populations consisting of 95% CD34⁺ cells or greater.

For competitive transplantation studies (FIGS. 4 and 5), venous angioblasts were prepared from HES2-RFP cells as previously described (Gage et al., 2020, Cell Stem Cell, 27:254-69) following mesoderm specification (10BOA3C: 10 ng/ml BMP4, 0 ng/ml Activin A, 5 ng/ml rhbFGF from day 0-4 and 3 µM CHIR99021 on day 2-4) and venous angioblast specification (30F10V10G: 30 ng/ml bFGF, 10 ng/ml VEGF-A, 10 µM GSI L-685-458 on day 4-8) in suspension EB format. Hybrid protocols (arm 1 and 2) designed to compare developmental stages included a “10B0A3C - Monolayer” and “12B4A - EB” version using HES2-RFP hESCs. In “10B0A3C - Monolayer”, day 4 mesoderm cells from the 10B0A3C published protocol were dissociated, counted and replated on GFR-Matrigel coated 10 cm plates for monolayer venous endothelial specification in “base media” supplemented with rhbFGF (30 ng/ml) and VEGF-A (10 ng/ml). In “12B4A - EB”, day 4 mesoderm cells from a rhbFGF (5 ng/ml), BMP4 (12 ng/ml), and Activin A (4 ng/ml) day 14 induction were not dissociated and were re-plated in 10 cm suspension EB culture following a 1:2 split in venous angioblast specification conditions (30F10V10G: 30 ng/ml bFGF, 10 ng/ml VEGF-A, 10 µM GSI L-685-458). All day 8 cells were purified by CD34 targeted MACS from dissociated single cell suspensions as described previously (Gage et al., 2020, Cell Stem Cell, 27:254-69) and above.

### Example 4-Flow Cytometry and Fluorescence Activated Cell Sorting (FACS)

In vitro derived single cell suspensions from day 4 EBs (Trypsin-EDTA, 5 min, FCS stopped), day 8 monolayers (PBS washed, TrypLE, 10 min), day 8 EBs (Trypsin-EDTA, 5 min, 0.2% w/v Collagenase Type 1, 60 mins, 37° C.) were used for flow cytometric staining using the following pre-conjugated antibodies: anti-KDR-biotin (R&D, Clone 89106, 15:100), Streptavidin-PeCy7 (BD Biosciences, 1:100) Streptavidin-BV421 (BioLegend, 1:100), anti-CD235a/b-APC (BD Biosciences, Clone GA-R2/HIR2, 2:100), anti-CD34-PeCy7 (EBiosciences, Clone 4H11, 1:100), anti-CD31-APCCy7 (BD Biosciences, Clone WM59, 1:100), anti-CD31-FITC (BD Biosciences, Clone WM59, 15:100) anti-CD184-BV421 (BioLegend, Clone 12G5, 1:100), anti-CD73-APC (BD Biosciences, Clone AD2, 0.25:100). In vitro surface marker staining for 20-30 minutes at 4° C. in FACS buffer (PBS without Mg/Ca with 5% FCS (Wisent), 0.02% NaN3 (SigmaAldrich), DNASE (1 U/ml)). Following liver dissociation and non-parenchymal cell recovery, cells (1×10⁶ cells / 100 µl) were treated with FcR blocking human IgG (Miltenyi, 15:100) in RPMI1640 with 0.5% BSA and 1 U/ml DNAse along with primary and secondary antibodies: anti-CD31-APCCy7 (BD Biosciences, Clone WM59, 1:100), rabbit-antiLYVE1 (Abcam, Polyclonal, 1:1000), and anti-CD32-PeCy7 (BioLegend, Clone FUN-2, 3:100), Donkey-anti-rabbit-APC (F′(ab)2 fragment, Jackson, 0.5:100). For cell sorting and engraftment analysis, samples were stained in IMDM with 0.5% FCS, DNASE (0.1 U/ml), and DAPI (Biotium, 0.1 µg/ml) before analysis using a LSRII flow cytometer (BD) or a Fortessa flow cytometer equipped with a 532 nm green laser (BD) or sorting with a FACSAriaIII (BD) instrument (532 nm laser) at the SickKids/UHN flow cytometry facility. Data were analyzed using FlowJo software (version 10.6.1, Tree Star).

### Example 5-Quantitative Reverse Transcription PCR

Total RNA was isolated from bulk, MACS-enriched, or FACS-purified in vitro or in vivo hPSC-derived samples using RNAqueous-micro kit (Invitrogen) with post column DNAse treatment. cDNA synthesis (iSCRIPT, BioRad) was performed using up to 1 µg of RNA subsequent RT-qPCR was performed using a CFX384 Touch Real-Time instrument (BioRad), QuantiFast SYBR Green PCR kit (Qiagen) or SsoAdvanced Universal SYBR® Green Supermix (BioRad) and primers described in Table 1, following manufacture recommendations. Gene expression relative to TBP was determined from technical duplicates evaluated for relative copy number, reaction efficiency, and genomic DNA contamination (<0.01% of TBP content) using a 10-fold dilution series of sonicated human genomic DNA standards made in house from wild type HES2 hESC cells ranging from 2.5 pg/µl to 25 ng/µl. Heatmaps and bar graphs of gene expression were generated in Prism version 9 (Graphpad) with the exception of FIG. 2G which generated manually in Microsoft Excel.

TABLE 1

Primers for Quantitative Reverse Transcription PCR.

| Gene Target | Sequence (5′-3′) | Orientation (FW RV) | SEQ ID NO |
|---|---|---|---|
| ACTA2 | TCCGGGACATCAAGGAGAAACTGT | FW | 1 |
| ACTA2 | TTCCGATGGTGATCACTTGCCCAT | RV | 2 |
| ALPNR | GCATGGAGGAAGGTGGTGATTT | FW | 3 |
| ALPNR | CAACATGTAGATGGCAGGGATGAG | RV | 4 |
| CALCRL | GCTGTGAGAGCTACTCTTATCTTG | FW | 5 |
| CALCRL | GTAGTCATATACCTCCTCTGCAATC | RV | 6 |
| CD14 | TCAACCTAGAGCCGTTTCTAAAG | FW | 7 |
| CD14 | TACCAGTAGCTGAGCAGGAA | RV | 8 |
| CD36 | ACACTAATTCACCTCCTGAACAA | FW | 9 |
| CD36 | GGTCTCCAACTGGCATTAGAATA | RV | 10 |
| CXCR4 | AGGGAACTGAACATTCCAGAGCGT | FW | 11 |
| CXCR4 | AAACGTTCCACGGGAATGGAGAGA | RV | 12 |
| ETV2 | CCGGGCATGAATTACGAGAA | FW | 13 |
| ETV2 | CGAAGCGGTACGTGTACTTT | RV | 14 |
| EPHB4 | GTCGTCACCACCAAACTCAA | FW | 15 |
| EPHB4 | GGGAACGGGGAGAAAAATTA | RV | 16 |
| F8 | CAGGAGGGTGCATCCAATTTA | FW | 17 |
| F8 | GCAGGTTTCTCCTCACTTCTT | RV | 18 |
| FCGR2B | CCTGATGACCAGAACCGTATTT | FW | 19 |
| FCGR2B | TACCAGATCTTCCCTCTCTGATT | RV | 20 |
| FOXF1 | CCGAAAGGAGTTTGTCTTCTCT | FW | 21 |
| FOXF1 | AAGGCTTGATGTCTTGGTAGG | RV | 22 |
| GATA4 | TCTGGAGGCGAGATGGG | FW | 23 |
| GATA4 | GCGCTGAGGCTTGATGAG | RV | 24 |
| gDNA | CAACATACCTCATAGCATTATACAAGAC | FW | 25 |
| gDNA | CCCTATGTTCCTGGTTCTTCATATT | RV | 26 |
| GYPA | AACTGTGTCGGAGCACTCACTGAA | FW | 27 |
| GYPA | CTGGATGTCCGGTTTGCACATCTT | RV | 28 |
| GYPB | CACCTGCTGTTCTCTTGTTTATG | FW | 29 |
| GYPB | CAGTAATAGTGAGGCAGGAGAAC | RV | 30 |
| GYPC | GTCCTAGTCTCCCTCCTCTTC | FW | 31 |
| GYPC | TCTGCACTCTCAGCAAACTC | RV | 32 |
| GYPE | AGACTCCAGTGAATCGCTTTC | FW | 33 |
| GYPE | GATCTCTTTGCAGGGCTATGT | RV | 34 |
| HAND1 | CCAGACGCAGGAAGATGAAA | FW | 35 |
| HAND1 | GAGAGACAGAGAAAGAGAGAAAGTG | RV | 36 |
| HAND2 | CCGAAAGGAGAGGATCTGAGAA | FW | 37 |
| HAND2 | TTTAGCTGCGAGTAACGTGTC | RV | 38 |
| KDR | AGTCTGTGGCATCTGAGGGCTCAA | FW | 39 |
| KDR | AGTACACGGTGGTGTCTGTGTCAT | RV | 40 |
| LYVE1 | CCTACTACTACTCCTCCTGCTC | FW | 41 |
| LYVE1 | TGTAGACATGGTGCTAGTTTCC | RV | 42 |
| MESP1 | AGCCCAAGTGACAAGGGACAACT | FW | 43 |
| MESP1 | AAGGAACCACTTCGAAGGTGCTGA | RV | 44 |
| MKI67 | CTGAGGCAGAACAGCAAATA | FW | 45 |
| MKI67 | AGGTCTTCATGGGCTTCT | RV | 46 |
| MSGN1 | GTGTGTATATGTGTGTGTGTTTGT | FW | 47 |
| MSGN1 | CTGCCTGTGACTTTCCATTTATC | RV | 48 |
| MRC1 | TCATCATTGTGATCCTCCTGATTT | FW | 49 |
| MRC1 | CTGGGCTTGACTGACTGTTAAA | RV | 50 |
| NFATC1 | CCTGCTGCCTTACACAGTGCATTT | FW | 51 |
| NFATC1 | TGCGACTATGAACAAGCCTACGGT | RV | 52 |
| NR2F2 | TGATGTAGCCCATGTGGAAAG | FW | 53 |
| NR2F2 | GCTGCCGGACAGTAACATATC | RV | 54 |
| NRP2 | CTTTATCGAGATTCGGGATGGG | FW | 55 |
| NRP2 | CGGAGGTGAACTTGATGTAGAG | RV | 56 |
| NT5E | CACACGGCATTAGCTGTTATTT | FW | 57 |
| NT5E | AGGGACAAGTGCAGGTTTAG | RV | 58 |
| OSR1 | CACCAACTACTCCTTCCTTCAG | FW | 59 |
| OSR1 | ACGCGCTGAAACCATACA | RV | 60 |
| PAX3 | AAGAGGAAACAGCGCAGAAG | FW | 61 |
| PAX3 | GGTCAGTTCCTCCCTAGTATAA | RV | 62 |
| PDGFRA | TCCTCTGCCAGCTTTCATTAC | FW | 63 |
| PDGFRA | CCTGCCTTCAAGCTCATTCT | RV | 64 |
| PECAM1 | TTCCTGACAGTGTCTTGAGTGGGT | FW | 65 |
| PECAM1 | TTTGGCTAGGCCTGGTTCTCATCT | RV | 66 |
| PLVAP | GGACAAGGACAAGTTTGAGATGG | FW | 67 |
| PLVAP | TAGAGGTTGTAACCCAGGTTGT | RV | 68 |
| RAMP3 | TACTATGAGAGTTTCACCAACTGC | FW | 69 |
| RAMP3 | GAGAAGAACTGCCTGTGGATG | RV | 70 |
| STAB2 | CTGACCCTTTGGCTCTTCTT | FW | 71 |
| STAB2 | GATACAAAGACGGAGGCTTACA | RV | 72 |
| TBP | TGAGTTGCTCATACCGTGCTGCTA | FW | 73 |
| TBP | CCCTCAAACCAACTTGTCACCAGC | RV | 74 |
| TBXT | TGTCCCAGGTGGCTTACAGATGAA | FW | 75 |
| TBXT | GGTGTGCCAAAGTTGCCAATACAC | RV | 76 |
| VWF | AGGGCCTGAAGAAGAAGAAGGTCA | FW | 77 |
| VWF | TAACGATCTCGTCCCTTTGCTGCT | RV | 78 |

List of single exon targeting primers used for RT-qPCR analysis in this study. Note that “gDNA” primers target the untranscribed upstream promoter element of PAGE1 (GAGEB1) serving to detect gDNA contamination in reverse transcribed cDNA samples.

### Example 6-Cell Transplantation in Neonatal and Adult NSG Mice

Cell transplantation from previously frozen day 8 cells was performed as described using an irradiated neonate or monocrotaline (MCT, SigmaAldrich) conditioned adult NSG models (Gage et al., 2020, Cell Stem Cell, 27:254-69). In the neonatal model, NSG P1-P4 pups were irradiated (100 cGy) 18-24 hours before direct intrahepatic injection of 1.5×10⁶ cells in 30 µl of base media by 30 G½ needle on a Hamilton syringe. In the adult model, 8-20 week old male and female NSG mice received a 150 mg/kg intraperitoneal MCT injection 18-24 hours before surgical cell delivery via intrasplenic injection of 1.5×10⁶ cells in 40-50 µl of base media. After cell therapy, mice were transitioned to enrofloxacin for 14 days and grafts were allowed to expand and mature for up to 171 days.

Competitive cell transplantation experiments were performed in MCT-conditioned adult male NSG mice. Cell mixtures were prepared from day 8 CD34⁺ venous cell populations with similar post-thaw viability above 85% DAPI⁻. On the day of transplantation, cells were thawed, counted and mixed in indicated defined ratios such that the total inoculum contained 1×10⁶ cells (for example 5×10⁵ GFP and 5×10⁵ RFP). Each inoculum was delivered to n=2-6 replicate mice via intrasplenic surgical delivery followed by 40-45 days of in vivo graft expansion and maturation.

Graft composition analysis from neonatal, adult or competitive adult NSG transplantation experiments was achieved by liver dissociation and non-parenchymal cell (NPC) isolation at the same time as heparinized cardiac blood sampling and histological sampling as previously described in detail (Gage et al., 2020, Cell Stem Cell, 27:254-69). Briefly, engrafted livers were mechanically and enzymatically dissociated to single cells by 40 minutes rotating in a 10 cm plate at 70 RPM (37° C., 5% CO₂, 95% Air) in 24 ml liver dissociation solution comprising (66% v/v HANKS buffer, 33% v/v RPMI1640, BSA (0.17% w/v, Sigma-Aldrich), collagenase type 1 (0.3125% w/v, Sigma-Aldrich) and DNAse (1 U/ml). NPC liver fraction, lysed of red blood cells was sampled for flow cytometric graft composition assessment with or without FACS mediated cell recovery for RT-qPCR analysis.

### Example 7-Cell Transplantation and Functional Assessment in NSG-HA Mice

Cell transplantation in NSG-HA mice was performed as described (Follenzi et al., 2008, J. Clin. Invest., 118:935-45; Olgasi et al., 2018, Mol. Ther. Methods Clin. Dev., 23:551-66). NSG-HA mice were treated with MCT (150 mg/kg) 18-24 hours before surgery. On the day of surgery, NSG-HA mice received 4 U of rhFVIII (Kovaltry, Bayer) intravenously to temporarily facilitate coagulation for surgery after which 1.5×10⁶ (cohort 1, HES2-GFP) or 3.0×10⁶ (cohort 2, HES2-RFP) day 8 CD34⁺ venous ECs derived from KDR⁺CD235a/b⁺ day 4 populations were delivered via intraportal or intrasplenic surgical delivery routes. Cohort 1 mice were terminated at 12 weeks post cell delivery and Cohort 2 mice were terminated at 16 weeks post cell delivery for FVIII antigen and histological analysis.

Functional FVIII activity in control and cell treated mice was assessed by activated partial thromboplastin time (aPTT) and tail clip bleeding time assays as previously described (Merlin et al., 2019, Blood Adv., 3:825-38). For longitudinal tracking aPTT assays, 3.2% citrated peripheral blood derived plasma (2000×g, 15 min) was compared to standard curves of serially diluted full length recombinant FVIII (Kovaltry) in pooled hemophiliac mouse plasma. Results are expressed as IU/ml where 1 IU/ml is 100% normal human FVIII activity benchmarked to the WHO international standard. For blood loss assays, the distal 2-2.5 mm of tail was cut off from anesthetized mice and blood was collected for 10 minutes in 14 ml of warmed (37° C.) saline with bleeding times recorded. Precipitated RBC containing blood cells were lysed and absorbance was measured at 575 nm, using a VictorX spectrophotometer (Perkin Elmer). Human specific FVIII antigen levels in NSG, and NSG-HA mice were determined by ELISA (Affinity Biologicals, FVIII-AG, Lots: AG80050 and AG8-0055) using heparinized cardiac puncture samples as previously described (Gage et al., 2020, Cell Stem Cell, 27:254-69) following manufacture recommended protocols with 1:1-1:2 diluted samples to reduce matrix effects. FVIII antigen levels are reported as IU/ml where 1 IU/ml represents 100% normal human antigen levels benchmarked to the WHO international standard.

### Example 8-Immunohistochemistry and Immunofluorescence

At various time points after transplantation (10-24 weeks), liver tissue samples were fixed in PFA (4%w/v, 24-48 hours, 4° C., Electron Microscopy Services), stored in ethanol (70% v/v, 1-30 days, Commercial Alcohols) followed by paraffin processing and immunohistochemical staining by the UHN Pathology Research Program as previously described (Gage et al., 2020, Cell Stem Cell, 27:254-69). Primary antibodies included: rabbit anti Ku80 (Cell Signaling, 2180, clone C48E7, 1/1000 overnight), rabbit anti human LYVE1 (Abcam, Ab36993, polyclonal, 1/1000 overnight), mouse anti human CD31 (DAKO, M0823, clone JC/70A, 1/50 overnight), goat anti human CD32B (NSJ Bioreagents, R34966, polyclonal, 1/1000 overnight), sheep anti human Stabilin 2 (R&D, AF3645, polyclonal, 1/1000 overnight), goat anti GFP (Rockland, 600-101-215, polyclonal, 2/100 overnight). Secondary kits and developing reagents, MACH4 (Inter Medico, BC-M4U534L), IMMPRESS-AP, (Vector, MP5401-15), developers (Inter Medico, BC-WR806H or DAKO, K3468) were used as per kit instructions. Sections were imaged at the Advanced Optical Microscopy Facility using a Scan-Scope AT2 (Aperio) slide scanner, 40x objective and associated software. Immunofluorescent confocal microscopy was performed on 4-5 µm thick liver sections following sodium citrate (pH 6.0) antigen retrieval and casein blocking (Dako). Overnight applied primary antibodies included: rabbit anti Ku80 (Cell Signaling, 2180, clone C48E7, 1/100), mouse anti FVIII (Abcam, AB41188, clone 27.4, 2/100), goat anti GFP (Rockland, 600-101-215, polyclonal, 2/100), mouse anti Ki67 (Dako, M7240, clone MIB-1, 1/100), rabbit anti SMA (Abcam, Ab32575, clone E184, 1/100), mouse anti Vimentin (Sigma-Aldrich, V6630, clone V9, 1/100), rabbit anti Collagen I (Abcam, Ab34710, polyclonal, 1/100), and rabbit anti Collagen III (Abcam, Ab7778, polyclonal, 1/100). Secondary antibodies (Invitrogen) including donkey anti host - AF488, AF555, or AF647 variants applied for 1-2 hours at room temperature with DAPI counterstain followed by washing and mounting (Prolong Diamond Antifade, Invitrogen). Immunofluorescent slides were imaged using a Leica SP8 confocal imaging system (405 nm, 488 nm, 552 nm, and 638 nm laser lines) through a HC PL APO CS2 63x / 1.40 NA oil objective lens and images were processed for presentation using ImageJ (FIJI) (Schindelin et al., 2012, Nat. Methods, 9:676-82).

### Example 9-Quantification and Statistical Analysis

All data are presented as mean ± standard error of the mean (SEM) with sample sizes (n) depicted as individual dots on bar graphs or specified in figure legends and represent biological replicates as either independent cell differentiations or mice. Different “Cohorts” use independent in vitro differentiation biological replicates. No statistical method was used to pre-determine the sample sizes and neither randomization nor investigator blinding was performed on samples. Statistical significance was determined in Prism 9 (Graphpad) software using students T-test, one-way ANOVA, or two-way ANOVA analysis with Bonferroni post-hoc test as indicated. Results were considered to be significant at p < 0.05 (*/^{#}), p < 0.01 (**/^{##}), p < 0.001 (***/^{###}) with specific comparisons indicated in the respective figure legend.

### Example 10-Mesodermal and Endothelial Specification of hPSCs

As we have shown in earlier studies that KDR⁺ mesoderm subpopulations separated based on the co-expression of CD235a/b display different hematopoietic and cardiovascular potential, we were interested in determining if LSEC potential would also differ between them. At the same time, we also aimed to improve the yield of venous endothelial cells by simplifying our endothelial specification to a monolayer format free from exogenous Notch inhibition. For these studies, hPSCs were differentiated using our well established embryoid body (EB)-based protocol that involves the formation of EBs (day 0-1), the induction of a primitive streak population (PS) and mesoderm (days 1-4) and the specification of mesoderm to an endothelial fate (days 4-8) (FIG. 1A). The day 8 populations were analyzed for the presence of ECs that displayed a venous phenotype: CD34⁺ CD31⁺ CD73⁺ CD184⁻. Using the strategy described in our previous studies (Kattman et al., 2011, Cell Stem Cell, 8:228-40; Lee et al., 2017, Cell Stem Cell, 21:179-94; Sturgeon et al., 2014, Nat. Biotechnol., 32:554-61), we varied the concentrations of BMP4 and Activin A (24 combinations) between days 1 and 4 of differentiation, with the goal of generating KDR⁺CD235a/b⁺ (K⁺2⁺) mesoderm that will efficiently give rise to VECs. Varying concentrations of BMP4 (B; 3-24 ng/ml) and Activin A (A: 0-16 ng/ml) induced subpopulations of mesoderm that expressed different sets of markers (FIGS. 1B-1I, 7, 9A-9I). The K⁺2⁺ target population was induced at different concentrations of factors and most efficiently when they were combined in a 3:2 ratio of BMP4 (B) to Activin A (A) (FIG. 1E: 3B2A, 6B4A, 12B8A, 24B16A). TBXT (Brachyury) expression, indicative of PS formation, was observed in populations induced in the absence of Activin A or with high concentrations of factor suggesting slowed relative development. The addition of Activin A at all concentrations enhanced cell numbers (10-30 fold) in the presence of all concentrations of BMP4 (FIGS. 1C, 9F). Populations induced with low concentrations of Activin A together with medium/high amounts of BMP4 (6, 12, 24 ng/ml) expressed the hemato-endothelial markers CD34 and ETV2 by day 4, a pattern indicative of accelerated onset of endothelial specification (FIGS. 1F, 1I).

To measure the venous endothelial potential of these mesoderm populations, we next dissociated the EBs at day 4 and re-plated the cells in a monolayer culture on Matrigel coated plates in a simplified venous endothelial specification media (10 ng/ml VEGF-A, 30 ng/ml bFGF) for 4 days. Total cell numbers were similar in all conditions (FIG. 9J). Endothelial (CD34⁺CD31⁺) commitment was most robust from the mesoderm induced with the 3B:2A ratio (FIGS. 1L, 9K). Cells with a VEC profile (CD34⁺CD31⁺CD73⁺CD184⁻), induced in the presence of low concentrations of VEGF-A (10 ng/ml), were generated from most mesoderm inductions with the exception of 3B2A. By contrast, arterial cells were specified by high concentrations of VEGF-A (100 ng/ml) from mesoderm induced with 12B4A (FIGS. 1J-1M, 8, 9M). By combining the total yield of day 4 K⁺2⁺ mesodermal cells and the day 8 VEC yield, we determined that the range of 6-12 ng/ml BMP4 and 4-8 ng/ml Activin A generated the highest number of VECs from K⁺2⁺ enriched mesoderm. Of these, mesoderm induced with 12 ng/ml BMP4 and 4 ng/ml Activin A consistently gave rise to the highest number of these VECs (FIGS. 1M, 9N) and therefore was chosen for all following experiments.

To optimize production of the target VEC population, we translated the protocol from 6-well plates to 10 cm dishes, a change that yielded 7-fold higher mesoderm, 5-fold high total cell numbers (day 8) and 11-fold more CD34⁺ cells (day 8) compared to our previously published venous angioblast production protocol (Gage et al., 2020, Cell Stem Cell, 27:254-69) (FIGS. 2A-F). These improvements resulted in an overall yield of 0.9 ECs for each input hPSC. Molecular comparison of mesoderm induced with the combination of 10 ng/ml BMP4, no Activin A, and 3 µM CHIR (10B0A3C) as detailed in our previously published protocol (Gage et al., 2020, Cell Stem Cell, 27:254-69) to the 12B4A-induced unsorted and K⁺2⁻, K⁺2⁺, K⁻2⁺ and K⁻2⁻ fractions revealed differences in the expression levels of genes associated with lateral plate (LPM) and paraxial/myogenic mesoderm. Specifically, 12B4A-induced unsorted and K⁺2⁻ and K⁺2⁺ fractions showed elevated expression of LPM genes including APLNR, FOXF1, HAND1, and HAND2 (Firulli et al., 1998, Nat. Genet., 18:266-70; Mahlapuu et al., 2001, Development, 128:155-66; Ormestad et al., 2004, Dev. Dyn., 229:328-33; Vodyanik et al., 2010, Cell Stem Cell, 7:718-29) and decreased expression of early intermediate and paraxial / myogenic genes such as OSR1, MSGN1, and PAX3 compared to the (10B0A3C)-induced mesoderm (Chalamalasetty et al., 2014, Development, 141:4285-97; James et al., 2006, Development, 144:2995-3004; Mugford et al., 2008, Dev. Biol., 324:88-98; Tani et al., 2020, Exp. Mol. Med., 52:1166-77) (FIGS. 2G, 10). FACS isolated K⁺2⁻ and K⁺2⁺ day 4 mesoderm subpopulations generated day 8 CD34⁺CD31⁺CD73⁺CD184⁻ VECs at the same frequency as the unsorted population (FIGS. 2D-2F). The sorted populations progressed through the expected developmental steps that included the sequential upregulation of the endothelial lineage markers ETV2 (day 5-6) and NFATC1 (day 5-8) (FIG. 2H). Analyses of day 8 MACS-enriched CD34⁺ cells from the sorted mesoderm populations showed they had the potential to generate endothelial cells that expressed markers indicated of venous lineage specification: NR2F2, NRP2, NT5E, and EPHB4 (Ditadi et al., 2015, Nat. Cell Biol., 17:580-91; Fish and Wythe, 2015, Dev. Dyn., 244:391-409; Zhang et al., 2017, PNAS USA, 114:E6072-8) (FIG. 2I) at similar levels to our previously observed venous angioblast derived VECs (Gage et al., 2020, Cell Stem Cell, 27:254-69). Together, these findings demonstrate that the modifications made during LPM induction and endothelial specification resulted in dramatic improvements in the efficiency of CD34⁺CD31⁺CD73⁺CD184⁻ VEC generation. Additionally, they show that isolated mesoderm subpopulations retain the capacity to generate VECs.

### Example 11-Engraftment and Maturation to LSECs in NSG Mice

To evaluate the potential of the day 8 VEC populations to generate mature functional LSECs, we transplanted them into either irradiated neonatal NGS pups or into monocrotaline (MCT) conditioned NSG adults as described (Gage et al., 2020, Cell Stem Cell, 27:254-69) (FIG. 3A). The day 8 CD34⁺ VECs generated from the day 4 unfractionated (UNS) or K⁺2⁺ sorted mesoderm populations engrafted the non-parenchymal cell (NPC) component of the livers of both the neonatal and adult transplanted mice at levels (>1%) that we previously found to produce readily detectable hFVIII (Gage et al., 2020, Cell Stem Cell, 27:254-69) (FIGS. 3B-3C). Although both cell populations showed engraftment, adult animals transplanted with K⁺2⁺derived VECs had the largest grafts (FIG. 3C). Flow cytometric analyses of grafts generated from the K⁺2⁺- derived VECs showed that in neonatal and adult recipients, the majority of the cells were CD31⁺CD32⁺LYVE1⁺, a phenotype indicative of LSEC development (FIGS. 3D-3E). Molecular analyses of FACS isolated GFP+ CD31 + populations revealed that the LYVE1+ CD32+ cells expressed elevated levels of genes indicative of the LSEC fate including FCGR2B, LYVE1, STAB2, F8, CD14, MRC1, CD36, and RAMP3 and reduced levels of the pan endothelial genes PECAM1, VWF, and CALCRL (FIG. 3F) (Gage et al., 2020, Cell Stem Cell, 27:254-69; Halpern et al., 2018, Nat. Biotechnol., 36:962-70; MacParland et al., 2018, Nat. Commun., 9:4383; Strauss et al., 2017, Sci. Rep., 7:44356). Similar profiles were observed in neonatal transplanted animals (data not shown). Consistent with these patterns, histological studies showed readily detectable Ku80⁺CD31⁺ human endothelium lining the mouse liver sinusoids. These cells co-expressed CD32B, LYVE1, STAB2 and FVIII (FIGS. 3G-3H) demonstrating that they were of the LSEC lineage. Human specific FVIII was detected in the plasma of both neonatal and adult recipients and by 17 weeks post-transplant, the levels in adult transplanted animals reached levels of ~15% (0.15 ± 0.02 IU/ml) of those found in normal human plasma (FIG. 3I). These circulating FVIII levels, if translatable to patients with severe Hemophilia A (<1% FVIII) is potentially therapeutic (Blanchette et al., 2014, J. Thromb. Haemostasis, 12:1935-9; Srivastava et al., 2013, Haemophilia, 19:e1-47; White et al., 2001, Thromb. Haemost. 85:560).

To establish the kinetics of engraftment, we analyzed transplanted mice at weekly intervals over the first 21 days and then at day 100. A low frequency of hPSC-derived GFP⁺ cells (0.14±0.02% of NPC) was detected one day following transplantation. This frequency increased to 2.19±0.62% by day 21 and to 38.6±1.5% by day 100 (FIGS. 11A-11B). Approximately half of the transplanted population acquired the LSEC marker profile (CD31⁺CD32⁺LYVE1⁺) by day 14 of engraftment and by day 21, most of the GFP⁺ cells displayed this profile. Molecular analyzes showed highest levels of MKI67 expression at day 14, suggesting that this stage contain the most proliferating cells. The expression levels of genes indicative of LSEC development and maturation increased with time of engraftment. Some (CD36, GATA4, LYVE1, STAB2, and MRC1) showed an early upregulation of expression whereas for others (FCGR2B, CD14, RAMP3, and F8) the levels of expression increased at the later time point, day 100 (FIG. 11D). As we already found that the engrafted cells expressed the genes indicative of maturation by day 40, (FIG. 3), we focused all subsequent studies at this time point and beyond.

To ensure that this protocol is adaptable to different hPSC lines, we applied it to the H1-GFP hESC line (FIG. 12) and the PGPC17-11 iPSC line (FIG. 13). Induction with 12 ng/ml BMP4 and 4 ng/ml Activin A resulted in efficient generation of KDR⁺CD235a/b⁺ mesoderm on day 4. VEC specification of the H1- and PGPC17-11derived mesoderm gave rise to populations of CD34⁺ cells that could be purified by MACS. Following transplantation (64-79 days) into MCT-treated adult NSG mice ECs from both lines showed sinusoidal localized engraftment of CD31⁺STAB2⁺CD32B⁺LYVE1⁺ LSECs. Molecular analyses of FACS isolated cells from these recipients revealed that the cells expressed a LSEC gene signature similar to that observed in HES2-derived LSECs. Mice engrafted with both H1-GFP and PGPC17-11 VECs had circulating human FVIII levels between 5 and 19 IU/ml (% normal human plasma FVIII antigen levels) correlating with graft size. Taken together, these findings show that the VECs generated from different hPSC lines using our new protocol efficiently engraft the liver sinusoids of recipient mice and differentiate to mature functional FVIII secreting LSECs.

### Example 12-Quantitative Competitive Transplantation of hPSC-Derived Venous Endothelium

The levels of engraftment (60% of NPC) achieved in this study are higher than the highest levels we reported previously (48±4%) (Gage et al., 2020, Cell Stem Cell, 27:254-69) suggesting that the day 8 VEC population produced with our modified protocol has greater LSEC potential than that generated with our original protocol. To be able to quantify LSEC engraftment, we established a competitive repopulation assay, similar to that used to quantify hematopoietic stem cell engraftment (Harrison, 1980, Blood, 55:77-81) (FIG. 4A). For this assay, we generated day 8 VEC populations from congenic HES2 hESCs engineered to express either tdRFP (R) or eGFP (G) (Irion et al., 2007, Nat. Biotechnol., 25:1477-82) using the protocol established in this study and transplanted them as mixtures (total of 1x10⁶ cells) in the following ratios; 99R:1G, 90R:10G, 50R:50G, 10R:90G, 1R:99G, into MCT-treated adult NSG mice. At 42-43 days post-transplant the livers were analysed for the presence of GFP and RFP cells by flow cytometry. As shown in FIGS. 4B and 4C, the ratios of GFP/RFP cells detected in the liver grafts accurately matched the GFP/RFP ratio of VECs in the pre-transplant mixtures indicating that the two cell types competed equally well in vivo. Flow cytometric analyses revealed that all grafts consist predominantly (>80%) of CD31⁺CD32⁺LYVE1⁺ human LSECs regardless of the graft size or hESC of origin (RFP or GFP) (FIG. 4D). The ability to detect LSEC progeny from mixtures containing 1% competitor cells (99:1) highlights the sensitivity of this assay. To examine the spatial relationship of engraftment during competitive transplantation we immunostained matched samples of the five RFP:GFP ratios for LYVE1 marking LSECs from both coloured lines, and GFP to distinguish GFP lineage cells (FIG. 14). Similar to flow cytometric analysis, engraftment matched the inoculum composition and 1% competitor cells were easily detected from either line. Interestingly, these rare cells were found to not be scattered but instead clustered as foci of engraftment suggesting a clonal origin with limited mixing between liver lobules. To demonstrate that this assay is not influenced by the endothelial population used, we also competed RFP and GFP day 8 venous angioblasts generated with our previous protocol (Gage et al., 2020, Cell Stem Cell, 27:254-69). For this comparison we used ratios of 50:50 RFP:GFP or 90:10 RFP:GFP day 8 venous angioblast cells and analyzed the livers ~40 days later. As observed in the above study, the ratio of GFP⁺ and RFP⁺ cells in the graft reflected the ratio of venous cells in the pre-transplant population. Flow cytometric analyses showed that the grafts were phenotypically LSECs (FIGS. 4E, 4F). While overall graft size was smaller compared to previous VEC grafts, the smaller graft size did not impact GFP/RFP ratio assessment (FIG. 4G). Taken together, these findings show that LSEC engraftment is competitive over a broad range of cell ratios and that ECs generated with the same protocol display equal repopulation potential.

Using this competitive assay, we next compared the engraftment potential of ECs generated with our new and old protocols. For this comparison, we transplanted equal mixtures (50:50) of our previously described venous angioblasts (RFP) and our K⁺2⁺derived VECs (GFP) (FIG. 4H). In two independent experimental cohorts, we observed a much higher proportion of GFP⁺ than RFP⁺ (FIG. 4H, G/R graft ratio: 43.1 and 57.0) cells in the liver 42-43 days following transplantation. In all cases, the engrafting cells expressed the LSEC set of markers (CD31⁺CD32⁺LYVE1⁺, FIG. 4I). These findings clearly demonstrate that venous cells generated with our new protocol display dramatically better engraftment than those produced with our original strategy.

As we made two major changes in our protocol, mesoderm induction and endothelial specification, we were next interested in determining which contributed to the improved engraftment potential. To address this, we tested the two stages of differentiation independently using the following experimental design. In the first arm, mesoderm induced with the two different protocols was specified to an endothelial fate using the monolayer strategy described in this study, while in the second arm, mesoderm induced with the new protocol was specified using either the EB approach of our earlier study or the monolayer format (FIG. 5A). As shown in FIG. 5B (arm 1), KDR⁺ mesoderm induced with our previous combination of factors, 10BOA3, contained very few CD235a/b⁺ cells whereas the majority of the 12B4A-induced mesoderm was K⁺2⁺. Following 4 days of monolayer specification culture, the 10BOA3C-induced KDR⁺ mesoderm (RFP) generated a small CD31⁺CD34⁺ venous population (FIG. 5F). The resulting EC population contained some CD73^{high} CD184^{mid} cells that may represent migratory venous tip cell endothelium (Hasan et al., 2017, Nat. Cell Biol., 19:928-40). By contrast, the 12B4Ainduced mesoderm (GFP), by contrast gave rise to a larger CD31⁺CD34⁺ population that consisted mostly of CD73⁺CD184⁻ VECs. Competitive transplantation of the two day 8 CD34⁺ isolated populations (50%:50%) revealed that VECs from 12B4A-induced mesoderm showed 10 to 20-fold better engraftment than those from 10B0A3C-induced mesoderm, indicating that the method of mesoderm induction is important (FIG. 5C). Comparing methods of endothelial specification (arm 2) showed that the 12B4A-induced mesoderm generated comparably sized CD31⁺CD34⁺ endothelial populations in either the EB or monolayer formats that showed similar engraftment potential (FIGS. 5D-E). Quantification of cell numbers at different stages revealed that the VEC yield/input hPSC from the 10B0A3C-induced mesoderm was as low in the monolayer format (FIG. 5F) as in the EB format (FIG. 2C). Similarly, the yield from the 12B4A-induced mesoderm was equally high in both endothelial specification formats (FIGS. 5G, 2C). These findings clearly demonstrate that the method of venous specification does not contribute significantly to the improved engraftment observed in our new protocol. Rather, they show that the improvement is solely due to the subtype of mesoderm induced.

To determine if the improved engraftment potential of the day 4 12B4A-induced mesoderm was restricted to the K⁺2⁺ fraction, we next competed VECs generated from K⁺2⁻ mesoderm generated from RFP-hPSCs to K⁺2⁺ generated from GFP-hPSCs (FIG. 5H). We also competed the K⁺2⁺-derived GFP⁺ VECs to VECs made from unfractionated mesoderm induced from the RFP-hPSCs (FIG. 5I). Analyses of the animals at 41 days post-transplant revealed that the K⁺2⁺- and K⁺2⁻-derived VECs competed equally well in LSEC engraftment. Similarly, the VECs generated from the unsorted mesoderm showed equal engraftment potential to those derived from the K⁺2⁺ mesoderm. Collectively, these findings indicate that the mesoderm induction conditions are a defining factor in the generation of a VEC population with optimal LSEC engraftment potential and that expression of CD235a/b correlates well with the induction of this mesoderm.

### Example 13-Venous Endothelial Cell Therapy Corrects Bleeding in NSG-Hemophilia A Mice

To formally test the feasibility of treating FVIII-deficient Hemophilia A with cell replacement therapy, we transplanted Hemophilia A NSG mice that lack FVIII activity (NSG-HA) (Zanolini et al., 2015, Haematologica, 100:881-92) with the day 8 hPSC-derived VECs. Two cohorts of animals were transplanted by direct injection either into the portal vein (intraportal, IP) or into the spleen (intrasplenic, IS). Analyses of the first cohort (1.5 × 10⁶ cells per mouse) over the 2-12 week monitoring period showed that the transplanted animals had human FVIII activity as measured by one-stage aPTT assay (0.011-0.035 IU/m1, 1.1-3.5% of normal human levels) standardized to Kovaltry (recombinant full length human FVIII) (FIG. 6A). The non-transplanted control NSG-HA mice did not show this activity. This level of human FVIII was able to significantly reduce the bleeding time of these animals at 12 weeks post transplantation in animals engrafted with both approaches (IP and IS) (FIG. 6B). The animals in cohort 2 that received double the number of VECs (3 × 10⁶ cells per mouse) either IP or IS similarly showed elevated hFVIII activity (0.024-0.052 IU/ml, 2.4-5.2% of normal human levels) over the 4-16 week monitoring period (FIG. 6C). These levels also reduced bleeding times in the recipients measured at the 16-week time point (FIG. 6D). Comparison of animals from cohort 1 and 2, that received intrasplenic transplants showed a cell dose related non-linear increase in hFVIII activity and circulating hFVIII antigen (FIGS. 6A, 6C, 6E). Histological analyses showed that human FVIII antigen was localized to Ku80⁺GFP⁺ human cells lining the liver sinusoids of the engrafted animals (FIG. 6F). Further characterization revealed that these human cells expressed markers indicative of an LSEC fate including CD31, LYVE1, CD32B and STAB2 (FIG. 6H). Ku80⁺Ki67⁺ cells were found to be lining the sinusoids demonstrating the presence of proliferating cells at 16 weeks post-transplant (FIG. 6G). Some animals from Cohort 2 had small nodules containing mesenchymal cells (Ku80⁺CD31⁻LYVE1CD32B⁻STAB2⁻) characterized by the expression of smooth muscle actin, vimentin, collagen I, and collagen III (FIG. 15). The presence of these nodules indicates that rare, non-endothelial progenitors contaminate the CD34⁺ VEC populations or derive from the engrafted endothelium in vivo. Similar mesenchymal cell nodules were detected in some of the grafts in our previous study (Gage et al., 2020, Cell Stem Cell, 27:254-69). Taken together, these findings show that hPSC VEC-derived engrafted LSECs can produce sufficient FVIII to correct the hemophilic bleeding phenotype in a pre-clinical mouse model of Hemophilia A, demonstrating the potential of a liver targeted cell therapy approach to treat this disease.

It is to be understood that, while the methods and compositions of matter have been described herein in conjunction with a number of different aspects, the foregoing description of the various aspects is intended to illustrate and not limit the scope of the methods and compositions of matter. Other aspects, advantages, and modifications are within the scope of the following claims.

Disclosed are methods and compositions that can be used for, can be used in conjunction with, can be used in preparation for, or are products of the disclosed methods and compositions. These and other materials are disclosed herein, and it is understood that combinations, subsets, interactions, groups, etc. of these methods and compositions are disclosed. That is, while specific reference to each various individual and collective combinations and permutations of these compositions and methods may not be explicitly disclosed, each is specifically contemplated and described herein. For example, if a particular composition of matter or a particular method is disclosed and discussed and a number of compositions or methods are discussed, each and every combination and permutation of the compositions and the methods are specifically contemplated unless specifically indicated to the contrary. Likewise, any subset or combination of these is also specifically contemplated and disclosed.

What is claimed is:

1. A method of making mesodermal cell-derived venous endothelial cells (VECs), comprising:
providing human pluripotent stem cells (hPSCs) having a pluripotent phenotype indicative of human induced pluripotent stem cells (iPSCs) or human embryonic stem cells (hESCs);
contacting the hPSCs with modulators of BMP and Activin signaling under appropriate conditions to produce mesodermal cells having a phenotype of KDR+ and CD235a/b+; and
inducing the mesoderm cells having the phenotype of KDR+ and CD235a/b+ to differentiate into mesodermal cell-derived VECs.

2. The method of claim 1, wherein the contacting step is performed in embryoid bodies (EBs) or in monolayer.

3. The method of claim 1, wherein the modulator of BMP signaling is selected from BMP4, BMP2, or small molecule BMP signaling agonists (e.g., ventromorphins [PMID 28787124], ID1 or ID2 [PMID 23527084], chromenone 1 [PMID 35108017], SB 4 [CAS number 100874-08-6] or similar).

4. The method of claim 1, wherein the modulator of Activin signaling is selected from Activin A, NODAL, or small molecule TGFbeta signaling agonists.

5. The method of claim 1, wherein the mesoderm cells further have a phenotype of PDGFRa+, CD56+ and APLNR+.

6. The method of claim 1, wherein the mesodermal cell-derived VECs have a phenotype of CD34+, CD31+, CD73+ and CD184-.

7. The method of claim 6, wherein the mesodermal cell-derived VECs further have a phenotype of NRSF2+, NRP2+, NT5E+ and EPHB4+.

8. The method of claim 1, wherein the inducing step is performed in embryoid bodies (EBs) or in monolayer adherent form.

9. The method of claim 1, wherein the inducing takes place in the presence of VEGF-A, VEGF-B, VEGF-C, VEGF-D or PIGF, or small molecule VEGFA signaling agonists.

10. The method of claim 1, wherein the inducing takes place in the presence of bFGF/FGF2 or small molecule FGF signaling agonists.

11. The method of claim 1, wherein the VECs engraft in vivo and mature to liver sinusoidal endothelial cells (LSECs).

12. The method of claim 1, wherein the VECs differentiate into functional liver sinusoidal endothelial cell like cells (LSEC-LCs) having a phenotype of CD31+, CD32+ and LYVE1+.

13. The method of claim 11, wherein the VEC-derived-LSECs further have a phenotype of CD32B+, STAB2+ and FVIII+.

14. The method of claim 11, wherein the LSECs express FCGR2B, LYVE1, STAB2, F8, CD14, MRC1, CD36 and RAMP3 at amounts that are greater than VEC-derived non-LSEC endothelium with a phenotype of CD31+ CD32- LYVE1-.

15. The method of claim 11, wherein the LSECs express PECAM1, VWF and CALCRL at amounts that are less than VEC-derived non-LSEC endothelium with a phenotype of CD31+, CD32- LYVE1-.

16. The method of claim 1, wherein the mesoderm cells generate a population of VECs that exhibits at least a 20-fold greater engraftment potential than venous angioblast cells derived from mesoderm cells having a phenotype of KDR+ and CD235a/b-.

17. A method of treating a subject suffering from a liver disease, comprising:
administering or delivering the cells made by the method of claim 1 to the subject, wherein the cells efficiently engraft in the liver, thereby treating the subject suffering from the liver disease.

18. The method of claim 17, wherein the liver disease is Hemophilia A or other monogenic endothelial disease.

19. The method of claim 17, wherein the liver disease is acute drug liver injury (e.g., Sinusoidal Obstruction Syndrome (human monocrotaline toxicity), or Acetaminophen overdose), chronic liver injury (e.g., NASH, NAFLD, Cirrhosis, chronic drug injury), liver cancer (e.g., primary, hepatocellular carcinoma; or secondary (e.g., colon, breast, pancreatic) liver metastatic cancer).

20. The method of claim 17, wherein the engrafted cells differentiate into LSECs.

21. The method of claim 20, wherein the LSECs express therapeutic amounts of FVIII.

22. The method of claim 20, wherein the LSECs are able to produce sufficient levels of FVIII to reverse the severe bleeding phenotype in a subject suffering from Hemophilia A.

23. The method of claim 17, wherein the administering or delivering step is repeated more than once.

24. The method of claim 17, wherein the administering or delivering step comprises intravenous delivery.

25. The method of claim 24, wherein the intravenous delivery is via infusion via the hepatic portal vein.

26. The method of claim 17, wherein the hPSCs, mesoderm cells, VECs or LSECs are rendered hypoimmunogenic through genetic addition or removal of immunoregulatory antigens resulting in cell populations that are not readily rejected by the recipient despite incomplete immunological matching.

27. The method of claim 17, wherein the method slows or reverses disease progression.
